(19) United States
(12) Patent Application Publication
(10) Pub. No.: US 2003/0003485 A1
(43) Pub. Date: Jan. 2, 2003

(54) Methods for identifying antigens

(75) Inventors: Akiko Uenaka, Okayama (JP); Eiichi Nakayama, Okayama (JP)

Correspondence Address: John R. Van Amsterdam, Wolf, Greenfield & Sacks, P.C., Federal Reserve Plaza, 600 Atlantic Avenue, Boston, MA, 02210, US

(73) Assignee: Ludwig Institute for Cancer Research, New York, NY

(21) Appl. No.: 10/145,396

(22) Filed: May 14, 2002

Related U.S. Application Data
(60) Provisional application No. 60/291,125, filed on May 15, 2001.

(51) Int. Cl. C12Q 1/68; G01N 33/567

(52) U.S. Cl. 435/6; 435/7.21

(57) ABSTRACT

Methods for identifying antigens using enzyme-linked immunospot assays are provided. The methods provide increased sensitivity, speed and reliability. The invention also relates to nucleic acids and encoded polypeptides which are cancer-associated antigens. The invention also relates to agents which bind the nucleic acids or polypeptides. The nucleic acid molecules, polypeptides coded for by such molecules and peptides derived therefrom, as well as related antibodies and cytolytic T lymphocytes, are useful, inter alia, in diagnostic and therapeutic contexts.

### RELATED APPLICATIONS

This application claims priority under 35 U.S.C. §119 from U.S. provisional application serial No. 60/291,125, filed May 15, 2001.

### FIELD OF THE INVENTION

The invention relates to methods for identifying antigens recognized by cells, particularly cytotoxic T-lymphocytes.

### BACKGROUND OF THE INVENTION

The identification of tumor antigens that elicit an immune response in the autologous host has long been an objective in tumor immunology (Boon and Old Curr. Opin. Immunol. 9:681-683, 1997). Following the introduction of methodology for cloning genes coding for T cell recognized antigens (Van den Eynde et al., J. Exp. Med. 173:1373-1384, 1991; van der Bruggen et al., Science 254:1643-1647, 1991), a range of human tumor antigens recognized by T cells has been identified (Brichard et al., J. Exp. Med. 178:489-495, 1993; Boon et al., Ann. Rev. Immunol. 12:337-365, 1994; Kawakami et al., Proc. Natl. Acad. Sci. USA 91:6458-6462, 1994; Rosenberg, Immunity 10:281-287, 1999; Chaux et al., J. Exp. Med. 189:767-778, 1999; Wang et al., J. Exp. Med. 189:1659-1668, 1999). There are several methods currently employed for identifying antigens recognized by cytotoxic T-lymphocytes (CTL). They include i) T-cell screening of transient transfectants of cDNA expression libraries (Van den Eynde et al., J. Exp. Med. 173:1373-1384, 1991; van der Bruggen et al., Science 254:1643-1647, 1991; Kawakami et al., Proc. Natl. Acad. Sci. USA 91:6458-6462, 1994), ii) amino acid sequence analysis of the peptide eluted from cell surface by acid with (Hunt et al., Science 255:1261-1263, 1992; Cox et al., Science 264:716-719, 1994) or without (Rotzschke et al., Nature 348:252-254, 1990; Uenaka et al., J. Exp. Med. 180:1599-1607, 1994) combining mass spectrometry, iii) peptide motif analysis of the targeted protein in the cell (Fisk et al., J. Exp. Med. 181:2109-2117, 1995; Jager et al., J. Exp. Med. 187:265-270, 1998), and iv) T-cell screening of synthesized peptide library based on the motif residues (Gundlache et al., J. Immunol. Methods 192:149-155, 1996; Munz et al., J. Immunol. 162:25-34, 1999). Within the foregoing, the former two methods, i.e., expression cloning of cDNA libraries and peptide elution, have been generally used as well-established methods especially for identifying antigens recognized by T cells on which no molecular information is available.

However, there are some intrinsic difficulties in those methods. In expression cloning of cDNA libraries, the number of recombinant plasmids in a pool should be as small as less than 100, sometimes less than 50, when sensitivity of CTL is low. Therefore, if 1×10⁵ recombinant plasmids are to be screened, minimum of 1,000 recombinant plasmid pools should be screened. Assay is done by detection of cytokines such as TNFα and IFNγ released in the culture supernatant. Purification of plasmids from a large number of bacterial pools, transfection of cells with the purified plasmids, and T-cell assay of cytokines to those transfectants are not an easy task and are time consuming. On the other hand, in peptide elution, it is practically impossible to have a fraction containing a single peptide even by repeated high performance liquid chromatography (HPLC) purification using columns, with various phases (Shastri, Curr. Opin. Immunol. 8:271-277, 1996). As a consequence, it is extremely difficult to read out the peptide sequence from contaminating signals.

An adaptation of the ELISPOT method for the identification of reactive T cells in a biological sample was described in U.S. Pat. No. 5,750,356. In this method, however, the T cells were required to be contacted with an antigen and cultured for an extensive period of time to permit expansion of the T cells in the sample. According to the method described in U.S. Pat. No. 5,750,356, the time period for T-cell expansion is typically greater than 3 days, and could be 5-7 days or as high as 10-14 days. Such long expansion phases frequently require the addition of to cytokines or growth factors to facilitate continued T-cell expansion and are susceptible to contamination and operator error. Consequently, the assays described in U.S. Pat. No. 5,750,356 are not preferred for rapid identification of antigens.

Moreover, in the methods described in U.S. Pat. No. 5,750,356, the identity of the T cell antigen was known, and the unknown factor was the presence of specific T cells reactive with the known antigen. Thus the identification of unknown antigens was not described in U.S. Pat. No. 5,750,356.

Due to the technical difficulties with the present methods as described above, there is a need for a simpler and more sensitive method for identifying antigens recognized by CTLs.

### SUMMARY OF THE INVENTION

The invention provides an improved method of identifying antigens recognized by CTLs using the ELISPOT assay. This method overcomes the disadvantages of classical cDNA expression cloning and other methods described above.

According to one aspect of the invention, methods for identifying a nucleic acid molecule encoding an epitope that specifically binds to a T cell receptor on a T cell when presented by a HLA molecule are provided. The methods include providing a T cell having a T cell receptor that binds the epitope, and providing a population of antigen presenting cells containing a library of nucleic acid molecules, wherein the cells express HLA molecules that present the epitope. The methods also include coculturing the antigen presenting cells with the T cell for a time sufficient for the T cell receptor to bind an epitope encoded by the library of nucleic acid molecules, detecting a factor secreted by the T cell in response to the T cell receptor binding using an ELISPOT assay, and correlating the secretion of the factor with the presence of the nucleic acid molecule encoding the epitope in the library.

In certain embodiments, the library of nucleic acid molecules is provided in an expression vector.

According to another aspect of the invention, method for identifying an antigen that specifically binds to a T cell receptor on a T cell are provided. The methods include providing a T cell having a T cell receptor that binds the antigen, and providing a population of candidate antigens. The methods also include coculturing the candidate antigens with the T cell for a time sufficient for the T cell receptor to bind an antigen, detecting a factor secreted by the T cell in response to the T cell receptor binding using an ELISPOT assay, and correlating the secretion of the factor with the presence of the antigen.

In some embodiments, the candidate antigens are presented by antigen presenting cells. In other embodiments, the candidate antigens are peptides. In some of these embodiments, the peptides are a library of random or semi-random peptides, while in other of these embodiments, the peptides are peptides derived from an antigenic protein. In certain of the foregoing embodiments, the antigen presenting cells are tumor cells. In other embodiments, the antigen presenting cells are cells infected with a microorganism.

In still other embodiments, the candidate antigens are presented as tetrameric complexes of HLA molecules and antigens.

In some embodiments of the foregoing methods, the step of detecting the presence of the secreted factor comprises capturing the secreted factor on a solid support. In still other embodiments of the foregoing methods, the antigen presenting cells are cocultured with the T cell for less than 36 hours, preferably for less than 24 hours, and more preferably for less than 18 hours.

In other embodiments of the foregoing methods, the secreted factor is interferon-y or tumor necrosis factor-α. In still other embodiments, the secreted factor is detected by binding to an immobilized capture antibody. Preferably capture antibody is immobilized on a membrane, and more preferably the membrane is a nitrocellulose membrane. In certain other embodiments, the secreted factor is detected by binding of a detection antibody. Preferably the detection antibody is conjugated to an enzyme. More preferably, the enzyme is selected from the group consisting of alkaline phosphatase, horseradish peroxidase and glucose oxidase.

According to a further aspect of the invention, a kit is provided, which includes a solid support for capturing factors secreted by T cells in response to antigen binding, a container containing a capture antibody that binds the secreted factors, a container containing a detection antibody that binds the secreted factors once bound to the capture antibody, and instructions for using the solid support, capture antibody and detection antibody for the idenfication of an antigen that is specifically bound by a T cell.

In some embodiments of the kit, the detection antibody is detectably labeled. In preferred embodiments, the detection antibody is labeled with an enzyme; in certain of such embodiments, the kit further includes a detectable enzyme substrate.

The invention also involves in some aspects, the surprising discovery that the ramp gene is expressed in mice bearing methylcholanthrene-induced fibrosarcomas. Thus, abnormally expressed ramp genes are recognized by the host's immune system and therefore can form a basis for diagnosis, monitoring and therapy. The mouse equivalent (i.e., homologs) of the human nucleic acid and polypeptide have also been identified as mouse ramp nucleic acid and polypeptide molecules.

The ramp molecules of the invention have not previously been described as being associated with cancer and no cancer-associated function was suspected for the human or mouse ramp molecules. The nature of the ramp genes as encoding antigens recognized by the immune system is, of course, unexpected. The invention thus involves in one aspect cancer-associated antigen polypeptides, genes encoding those polypeptides, functional modifications and variants of the foregoing, useful fragments of the foregoing, as well as diagnostics and therapeutics relating thereto.

According to yet another aspect of the invention, methods are provided for diagnosing and treating cancer. These methods include the use of mouse ramp nucleic acid and human ramp nucleic acid (SEQ ID NO:28), or fragments and variants thereof. The mouse ramp nucleic acid encodes an antigen precursor molecule, which comprises the amino acid set forth as SEQ ID NO: 11, or fragments and variants thereof. The invention also provides agents that bind the ramp nucleic acids or polypeptides, which are useful for diagnostic and/or therapeutic methods. The ramp nucleic acid molecules, polypeptides coded for by such molecules and peptides derived therefrom, as well as related antibodies and cytolytic T lymphocytes, are useful, inter alia, in diagnostic and therapeutic contexts and methods.

These and other aspects of the invention will be described in further detail in connection with the detailed description of the invention.

### BRIEF DESCRIPTION OF THE FIGURES

FIG. 1 shows the results of direct cytotoxicity (FIG. 1A) and IFNγ ELISPOT (FIG. 1B) assays of B-24 CTL against various target cells. Antibody blocking of cytotoxicity and IFNγ ELISPOT of B-24 are shown in FIG. 1C and FIG. 1D, respectively.

FIG. 2 depicts the results of an ELISPOT assay of B-24 CTL against CMS8 stimulator cells pulsed with pRL1a and two other L^{d} binding peptides (FIG. 2A) and to different numbers of pRL1a pulsed P1.HTR stimulator cells (FIG. 2B).

FIG. 3 shows assays using RLakt plasmids present at various amounts in a population of control GFP plasmids transfected into CMS8 cells. FIG. 3A depicts 96-well plate (small scale) ELISPOT assays at the indicated ratios, and FIG. 3B shows 24-well plate (large scale) ELISPOT assays at the indicated ratios. FIG. 3C and FIG. 3D show the results of IFNγ ELISA assays corresponding to the small and large scale ELISPOT assays of FIG. 3A and FIG. 3B, respectively.

FIG. 4 shows the 5 rounds of ELISPOT cloning of RLakt by B-24 CTL from a RL♂1 cDNA expression library, with progressively decreasing library pool size.

FIG. 5 depicts the analyses of the ELISPOT cloned nucleic acid insert. FIG. 4A shows that Sal I and Not I digestion of plasmid clone number 17 and 30 produced two bands: a 2.6 kb RLakt insert and a 3.0 kb vector fragment. FIG. 4B shows that PCR using RLakt specific primers produced the predicted 896-bp band.

FIG. 6. shows graphs of direct cytotoxicity (FIG. 6A) and IFNγ ELISPOT (FIG. 6B) of AT-1 CTL against various target cells and blocking of cytotoxicity (FIG. 6C) and IFNγ ELISPOT (FIG. 6D) of AT-1 CTL by various antibodies.

FIG. 7 depicts ELISPOT cloning of the antigen gene recognized by AT-1 CTL from a Meth A cDNA expression library. FIG. 7A, shows results of the first three rounds of screening in 10,000, 1,000 and 100 bacterial colonies per pool, which were done in large-scale assays using 24-well culture plates and the last two rounds of screening in 10 bacterial colonies and single clone, which were done in small-scale assays using 96-well culture plates.

FIG. 8 depicts deletion mutants and introduction of point mutations in S35. IFNγ production in the supernatant secreted by AT-1 CTL against various constructs was determined by ELISA after culture for 24 hr with transfected CMS8 cells. PCR fragments with deletion were amplified from S35 cDNA and cloned into pCI-neo vector with EcoR I and Sal I sites. Point mutations at nt 699 (A to C) and nt 701 (G to A) in Exp. III were introduced using site-directed mutagenesis kits.

FIG. 9 shows the identification of the epitope peptide recognized by AT-1. 9-mer overlapping peptides were synthesized in the ORF of the third reading frame (“3RF”) of S35. 1×10⁴ AT-1 cells were cultured with 1×10⁴ CMS8 cells in the presence of 1.0 μM synthetic peptides for 24 hr and IFNγ in the supernatant was measured by ELISA. H-2D^{d} peptide motifs are shown as XGPXXXXXL (SEQ ID NO: 26) and XGAXXXXXL (SEQ ID NO:27). The synthetic peptides are MERTPIQLGAEAIFRLVLM (SEQ ID NO:15); MERTPIQLG (SEQ ID NO:16); ERTPIQLGA (SEQ ID NO:17); RTPIQLGAE (SEQ ID NO:18); TPIQLGAEA (SEQ ID NO:19); PIQLGAEAI (SEQ ID NO:20); IQLGAEAIF (SEQ ID NO:21); QLGAEAIFR (SEQ ID NO:22); LGAEAIFRL (SEQ ID NO:10); GAEAIFRLV (SEQ ID NO: 23); AEAIFRLVL (SEQ ID NO:24); and EAIFRLVLM (SEQ ID NO:25).

FIG. 10 shows AT-1 CTL lysis (FIG. 10A) and IFNγ production (FIG. 10B) against synthetic LGAEAIFRL (SEQ ID NO:10) peptide-pulsed CMS8 cells. In FIG. 10A, CMS8 cells were ⁵¹Cr labeled and pulsed with the peptide at different concentrations. Cytotoxicity was determined by 4 hr ⁵¹Cr release assay. Effector to target cell ratio was 10. In FIG. 10B, 1×10⁴ AT-1 CTL was stimulated with 1×10⁴ CMS8 cells in the presence of the 1.0 μM peptide. IFNγ in the supernatant was measured by ELISA.

FIG. 11 depicts the EST database analysis. Top horizontal line represents nucleotide number of Meth A ramp homolog. Arrows represent EST sequences with homology to the Meth A ramp.

FIG. 12 depicts Exon 14 extension in Meth A. Exons and introns are shown as boxes and lines, respectively. Solid boxes are ORF. Arrow head shows the location of coding region for the antigenic epitope. The nt 12 of S35 cDNA corresponds to the first nt of the start codon in the extended exon 14 that is newly created and nt 722 corresponds to the first nt of the stop codon in exon 15. The nt 71 and 700 correspond to the splicing donor and acceptor sites, respectively. The mouse S35 sequence is provided as SEQ ID NO:13.

FIG. 13 shows the sequence alignment of the mouse (SEQ ID NO:11) and human (SEQ ID NO: 12) ramp proteins.

FIG. 14 depicts the genomic structure of the human ramp protein, and a comparison of the homology between the mouse and human cDNA sequences. * is the CTL epitope, LGAEAIFRL (SEQ ID NO: 10).

### DETAILED DESCRIPTION OF THE INVENTION

The invention described herein provides a marked improvement in sensitivity and efficiency of T-cell antigen cloning from cDNA expression libraries by using ELISPOT assays. The ELISPOT assay takes advantage of detecting the locally trapped cytokine produced by cells around a stimulator cell (Czerkinsky et al., J. Immunol. Methods 110:29-36, 1988). Combining large and small scale ELISPOT assays for expression cloning has the following advantageous features compared to conventional cDNA expression cloning using ELISA or bioassays for detection of cytokines produced by CTL in response to antigens.

First, the total number of recombinant plasmids that could be screened in a single well of a 24-well plate is more than 10,000 in a large scale ELISPOT cloning assay compared to less than 100 in a 96-well plate in IFNγ ELISA or TNFα bioassays used in conventional expression cloning.

Second, the total number of recombinant plasmids that can be screened in routine assay is 2×10⁵ in only a single 24-well plate in large scale ELISPOT cloning assay compared to 1×10⁵ recombinant plasmids in 10 96-well plates in IFNγ ELISA or TNα bioassays used in conventional expression cloning.

Thus the screening efficiency of ELISPOT cloning is roughly more than 200 times compared to that of classical expression cloning.

For cDNA expression cloning, the quality of the cDNA library is critical (Shastri, Curr. Opin. Immunol. 8271-277, 1996; Hess et al., Curr. Opin. Immunol. 10:125-130, 1998). For cDNA libraries prepared using either oligo (dT) or random primers, the number of clones in the library should be as large as possible and the insert size of the clones should be as long as possible. With prior methods, even with the use of cDNA libraries of sufficient quality, sorting positive clones from a large pool of clones in the screening process may have ambiguity due to weak signals (2, 3, 6). In addition, in conventional cloning methods one must account for variability in cytokine assays performed on different occasions during the prolonged screening period for a single library. Using the large scale ELISPOT assay described herein for cloning, a number of clones corresponding to a whole library was screened in a single 24-well plate assay. Screening this number of clones avoids misinterpretation of screening results with weak signals caused by background signal variation in each assay, and permits both evaluation of library quality and identification of positive signals with less ambiguity.

Sensitive T cells are required for detection of cDNA clones that code for antigenic epitopes. It can be difficult to maintain such responsive T cells stably (Sahin et al., Proc. Natl. Acad. Sci. USA 92:11810-11813, 1995). In the ELISPOT assays presented herein, small sized spots were observed for IFNγ low producer CTLs without reducing the number of spots. This property is beneficial for the use of T-cells having lower sensitivity.

The ELISPOT assays referred to herein are known to one of ordinary skill in the art. Briefly, ELISPOT assays (an acronym of enzyme-linked immunospot assays) involve the detection of a factor or factors secreted by a reactive T cell. A “reactive T cell” is defined herein as a T cell that secretes a factor in response to antigen binding, i.e., binding of an antigen by the T cell receptor. The secreted factors are captured as they are secreted on a solid phase in the area immediately surrounding the reactive T cell. Thus the assay concentrates the secreted factor in a limited area, thereby increasing the sensitivity of the assay and permitting ready identification of the number of reactive T cells. Cytokine ELISPOT assays are provided in Czerkinsky et al., J. Immunol. Methods 110:29-36, 1988; modifications are provided in the Examples below. Other modifications will by known to one of ordinary skill in the art (see, e.g., U.S. Pat. No. 5,750,356).

Antigens are presented to the T cells by HLA/MHC molecules. Antigens can be conveniently presented by several methodologies known to one of ordinary skill in the art. These include presentation by antigen presenting cells and complexes of HLA molecules and antigen. Antigen presenting cells include dendritic cells, B cells, CHO cells and COS cells, transfected as needed with HLA class I or HLA class II molecules and associated molecules required for antigen presentaion. Complexes include tetramers of HLA/MHC class I molecules. For example, several references describe methods in which fluorogenic tetramers of MHC class I molecule/peptide complexes were used to present antigen to specific CTL clones (Altman et al., Science 274:94-96, 1996; Dunbar et al., Curr. Biol. 8:413-416, 1998). Briefly, soluble MHC class I molecules are folded in vitro in the presence of β₂-microglobulin and a peptide antigen which binds the class I molecule. After purification, the MHC/peptide complex is purified and labeled with biotin. Tetramers are formed by mixing the biotinylated peptide-MHC complex with labeled avidin (e.g. phycoerythrin) at a molar ratio or 4:1. Tetramers are then contacted with a source of CTLs. The tetramers bind CTLs which recognize the peptide antigen/MHC class I complex. The use of MHC class II molecules as tetramers was recently demonstrated by Crawford et al. (Immunity 8:675-682, 1998). Multimeric soluble MHC class II molecules were complexed with a covalently attached peptide. The class II tetramers were shown to bind with appropriate specificity and affinity to specific T cells.

The terms “a factor secreted by the T cell” or “secreted factor” refer to proteins secreted by a T-cell in response to antigenic stimulation. A variety of secreted soluble factors can be detected by the assays disclosed herein. The soluble factors may be cytokines, lymphokines or chemokines. Typically this secreted factor is a lymphokine, such as enumerated below. As a result of the increased sensitivity of the assay, factors secreted by rare T-cells which occur in low frequency can be detected. Factors which are detected by this method include, but are not limited to lymphokines, cytokines and chemokines such as for example, interferon-γ (IFN-γ), tumor necrosis factor a (TNF-α), interleukins (IL) including IL-2, IL-3, IL-4, IL-10, IL-13 and granuloctye-macrophage colony stimulating factor (GM-CSF). Other suitable secreted factors will be known to one of ordinary skill in the art. As one of skill in the art will recognize, any secreted factor which can be detected by specific binding used in the subsequent assay detection step can be detected by this assay.

A variety of assay formats can be used to detect the increased levels of secreted factors produced by the assay described herein. Suitable assays include solid phase protocols. The assays can be run using competitive or non-competitive formats, and using a wide variety of labels, such as radioisotopes, enzymes, fluorescent compounds, chemiluminescent compounds, spin labels, and the like. It will be recognized that negative controls, i.e., samples run without added antigen, and positive controls, i.e., samples run with antigens, known to elicit factor secretion from T cells will be run as necessary under otherwise duplicative conditions to validate the assay results.

Some assays rely on heterogeneous protocols where a ligand complementary to the secreted factor (such as antibody against the secreted factor) is bound to a solid phase which is used to capture the secreted factor. The ligand may be conveniently immobilized on a variety of solid phases, such as dipsticks, particulates, microspheres, magnetic particles, test tubes, microtiter wells, and plastics, membranes (including nitrocellulose and nylon) and the like. The captured factor can then be detected using the non-competitive “sandwich” technique where a directly or indirectly labelled second ligand for the factor is exposed to the washed solid phase.

A commonly used assay format is the antibody capture assay. The general protocol involves the immobilization of a ligand, e.g., an unlabelled antibody for the secreted factor, on a solid phase; the secreted factor is allowed to bind to the immobilized antibody. The bound secreted factor is then detected by using a labelled secondary reagent that will specifically bind to the captured factor (“direct sandwich assay”). Alternatively, the secondary reagent will not be labelled, but will be detected by subsequent binding to labelled tertiary binding reagent complementary to the second binding reagent (“indirect sandwich assay”). The strength of signal from the bound label allows the determination of the amount of secreted factor present in the sample and this in turn allows the quantitation of the number of reactive T-cells reacting with antigen (e.g., as presented by antigen presenting cells) in the sample.

A variety of labelled secondary and/or tertiary reagents can be used to detect the presence of the bound secreted factor. Examples include, but are not limited to, anti-cytokine antibodies, anti-immunoglobulin antibodies, peroxidase/anti-peroxidase avidin/biotin complexes, protein A and protein G.

Detectable labels suitable for use in the present invention include any composition detectable by photochemical, biochemical, immunochemical, electrical, optical or chemical means. Useful labels in the present invention include biotin for staining with labeled streptavidin conjugate, fluorescent dyes and/or substrates (e.g., fluorescein, Texas red, rhodamine, green fluorescent protein, and the like), radiolabels (e.g., ³H, 25I, 35S, ¹⁴C, or ³²P), enzymes (e.g., horseradish peroxidase, alkaline phosphatase and others commonly used in an ELISA), colorimetric labels such as colloidal gold or colored glass or plastic (e.g., polystyrene, polypropylene, latex, etc.) beads and chemiluminscent labels. Patents teaching the use of such labels include U.S. Pat. Nos. 3,817,837; 3,850,752; 3,939,350; 3,996,345; 4,277,437; 4,275,149; and 4,366,241.

Means of detecting labels are well known to those of skill in the art. Thus, for example, radiolabels may be detected using photographic film, phosphorimager plates or scintillation counters, fluorescent or chemiluminescent markers may be detected using a photodetector or phosphorimager plates to detect emitted light. Enzymatic labels are typically detected by providing the enzyme with a substrate and detecting the reaction product produced by the action of the enzyme on the substrate, and colorimetric labels are detected by simply visualizing the colored label. Labels which are particularly useful in an ELISPOT assay as described herein are those which can produce a particulate product, such as when the combination of an enzyme and a substrate which gives a precipitating product. Combinations of such enzyme substrate pairs are alkaline phosphatase and 4-bromo-3-chloro indolyl phosphate/tetrazolium salts, naphthol AS-MX or napthol AS phosphate/Fast Blue BBN or Fast Red TR; horseradish peroxidase and 4-chloro-1-naphthol, 3,3′-diaminobenzidine (DAB), p-phenylenediamine, 3-amino-9-ethylcarbazole (AEC), 5,5′-tetramethylbenzidine and the like; glucose oxidase and t-nitroblue tetrazolium chloride (t-NBT)/m-phenazine methosulfate.

Following the identification of a nucleic acid in a library that encodes an epitope recognized by a specific T cell, one can clone the nucleic acid by sequential screening of smaller portions of the library until specific clones can be selected. Examples of this are described in the Examples, although other specific methods that vary from the presented method will be known to one of ordinary skill in the art.

If the methods are used to identify antigens by T cell binding of a peptide epitope from a random or semi-random library of peptides, then the peptide library is split into smaller portions in successive rounds of ELISPOT screening. By dilution and splitting of the library, fewer peptides will be screened in each successive round. Eventually a pure or nearly pure population of peptides, having a single major peptide, will be identified. The peptide sequence can be identified using routine means of amino acid sequence identification (e.g., direct sequencing, mass spectrometry).

Alternatively, an antigenic epitope can be identified by screening individual peptides or a defined mixture of peptides. For example, having identified an antigenic protein by screening of a library of nucleic acids as described herein, one may wish to identify the specific epitope recognized by the T cell. This can be accomplished by preparing an overlapping set of peptides that correspond to the entire sequence of the protein, and then screening the set of peptides using the ELISPOT methods described herein. Alternatively, one can make predictions as to the possible or probable epitopes using algorithms well known to one of ordinary skill in the art. A set of possible or probable epitope peptides then can be screened for T cell binding and production of secreted factors by the T cell.

As mentioned above, several methods are known in the art for predicting epitopes in amino acid sequences. One can search for HLA class I and HLA class II motifs using computer algorithms. For example, computer programs for predicting potential CTL epitopes based on known class I motifs has been described (see, e.g., Parker et al, J. Immunol. 152:163, 1994; D'Amaro et al., Human Immunol. 43:13-18, 1995; Drijfhout et al., Human Immunol. 43:1-12, 1995). Computer programs for predicting potential T cell epitopes based on known class II motifs has also been described (see, e.g Sturniolo et al., Nat Biotechnol 17(6):555-61, 1999). HLA binding predictions can conveniently be made using an algorithm available via the Internet on the National Institutes of Health World Wide Web site at URL http://bimas.dcrt.nih.gov. See also the website of: SYFPEITU: An Internet Database for MHC Ligands and Peptide Motifs (accessable via http://www.uni-tuebingen.de/uni/kxi/ or http://134.2.96.221/scripts/hlaserver.dll/EpPredict.htm).

Once a nucleic acid encoding an antigenic protein is identified by screening of a library, the amino acid sequence of the encoded protein can be deduced using standard codon tables, software that translates nucleotide sequence into amino acid sequence, etc. These methods are well known in the art. Full length or partial nucleotide or amino acid sequences can be used to query sequence databases to determine the identity of the antigenic protein. If the cloned sequence is not full length, then one can extend the sequence be comparison to sequence databases, or one can screen the same library or additional libraries (e.g., prepared from the cell or tissue of interest) to identify a full length sequence.

Through the use of methods of the invention as described herein, novel cancer-associated nucleic acid molecules and the cancer-associated polypeptides they encode have been identified. These cancer-associated molecules have been identified as ramp nucleic acids and polypeptides.

The invention involves the use of the ramp gene, a ramp protein encoded by a gene, a single functional fragment thereof, a single antibody thereto, etc. in diagnostic and therapeutic methods.

As will be clear from the following discussion, the invention has in vivo and in vitro uses, including for therapeutic, diagnostic, monitoring and research purposes. One aspect of the invention is the ability to fingerprint a cell expressing the ramp gene identified according to the invention by, for example, quantifying the expression of such gene products. Such fingerprints will be characteristic, for example, of the stage of the cancer, the type of the cancer, or even the effect in animal models of a therapy on a cancer. Cells also can be screened to determine whether such cells abnormally express the ramp gene identified according to the invention.

As used herein, a subject is a human, non-human primate, cow, horse, pig, sheep, goat, dog, cat or rodent. In all embodiments human cancer-associated antigens, which have substantial nucleotide and/or amino acid sequence identity to the presently identified cancer-associated antigens, and human subjects are preferred.

As used herein, an antigen precursor is substantially the full-length protein encoded by the coding region of an isolated DNA and the antigen is a peptide which complexes with MHC, preferably HLA, and which participates in the immune response as part of that complex. Such antigens are typically 9 amino acids long, for antigens presented by HLA class I molecules, although this may vary slightly.

The present invention in one aspect involves the cloning of cDNAs encoding cancer-associated antigen precursors. The sequences of human and mouse ramp cDNA and polypeptides identified according to the methods described herein are presented in the attached Sequence Listing. Human ramp nucleic acid (SEQ ID NO:28) and amino acid (SEQ ID NO:12) sequences correspond to Genbank Accession Nos: NM_{—}016448 and NP_{—}057532, respectively. The mouse ramp amino acid sequence is set forth as SEQ ID NO:11, and the the mouse S35 nucleic acid sequence is SEQ ID NO:13. The present invention in one aspect also involves a nucleic acid sequence of a cryptic exon in intron 14 (in between exons 14 and 15) of the ramp DNA sequence, and the antigenic peptide it encodes, which are useful in the diagnosis and treatment of cancer. The peptide antigen sequences of the invention include, but are not limited to, the peptide LGAEAIFRL (SEQ ID NO: 10), as well as longer peptides derived from ramp that includes SEQ ID NO:10.

Homologs and alleles of the ramp cancer-associated antigen nucleic acids of the invention can be identified by conventional techniques. Thus, an aspect of the invention is the nucleic acid sequences that code for the ramp cancer-associated antigen precursors. The ramp molecules of the invention include: (a) nucleic acid molecules which hybridize under stringent conditions to a ramp molecule consisting of a nucleic acid sequence that encodes the cancer-associated precursor set forth as SEQ ID NOs:11 and 12, (b) nucleic acid molecules that differ from the nucleic acid molecules of (a) in codon sequence due to the degeneracy of the genetic code, and (c) complements of (a) or (b). The invention also relates in part to fragments of the ramp cancer-associated nucleic acid, which codes for a polypeptide which, or a portion of which, binds an MHC molecule to form a complex recognized by an autologous antibody or lymphocyte. The invention also relates in part to ramp nucleic acid fragments that code for a polypeptide which, or a portion of which, binds to an MHC molecule to form a complex recognized by an autologous antibody or lymphocyte.

Identification of homologs of the cancer-associated antigens will be familiar to those of skill in the art. In particular, the methods described in Example 5 were effective in identifying the human homolog of ramp, as well as establishing it as a cancer-associated antigen useful in therapeutic and diagnostic applications for treatment and diagnosis of cancer. In general, nucleic acid hybridization is a suitable method for identification of homologous sequences of another species (e.g., human) which correspond to a known sequence (e.g., mouse cancer-associated sequences presented herein). Standard nucleic acid hybridization procedures can be used to identify related nucleic acid sequences of selected percent identity. For example, one can construct a library of cDNAs reverse transcribed from the mRNA of a selected tissue (e.g., breast, colon, or testis) and use the ramp cancer-associated antigen nucleic acids identified herein to screen the library for related nucleotide sequences. The screening can be performed at various stringencies to identify those sequences that are closely related by sequence identity, and more distantly related. Nucleic acids so identified can be translated into polypeptides and the polypeptides can be tested for activity. Identification of related sequences can also be achieved using polymerase chain reaction (PCR) and other amplification techniques suitable for cloning related nucleic acid sequences. Preferably PCR primers are selected to amplify portions of a nucleic acid sequence believed to be conserved (e.g., a catalytic domain, a DNA-binding domain, etc.). Again, nucleic acids are preferably amplified from a tissue-specific library. One also can use expression cloning utilizing the antisera described herein to identify nucleic acids which encode related antigenic proteins in humans or other species using the SEREX procedure.

The term “high stringency conditions” as used herein refers to parameters with which the art is familiar. Nucleic acid hybridization parameters may be found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. More specifically, stringent conditions, as used herein, refers, for example, to hybridization at 65° C. in hybridization buffer (3.5 × SSC, 0.02% Ficoll, 0.02% polyvinyl pyrrolidone, 0.02% Bovine Serum Albumin, 2.5 mM NaH₂PO₄(pH 7), 0.5% SDS, 2 mM EDTA). SSC is 0.15M sodium chloride/0.015M sodium citrate, pH 7; SDS is sodium dodecyl sulphate; and EDTA is ethylenediaminetetracetic acid. After hybridization, the membrane upon which the DNA is transferred is washed, for example, in 2× SSC at room temperature and then at 0.1-0.5× SSC/0.1× SDS at temperatures up to 68° C.

There are other conditions, reagents, and so forth which can be used, which result in a similar high degree of stringency. The skilled artisan will be familiar with such conditions, and thus they are not given here. It will be understood, however, that the skilled artisan will be able to manipulate the conditions in a manner to permit the clear identification of homologs and alleles of cancer-associated antigen nucleic acids of the invention (e.g., by using lower stringency conditions). The skilled artisan also is familiar with the methodology for screening cells and libraries for expression of such molecules which then are routinely isolated, followed by isolation of the pertinent nucleic acid molecule and sequencing.

In general homologs and alleles typically will share at least 85% nucleotide identity and/or at least 90% amino acid identity to the sequences of cancer-associated antigen nucleic acid and polypeptides, respectively, in some instances will share at least 90% nucleotide identity and/or at least 95% amino acid identity and in still other instances will share at least 95% nucleotide identity and/or at least 99% amino acid identity. The homology can be calculated using various, publicly available software tools developed by NCBI (Bethesda, Md.) that can be obtained through the internet (ftp:/ncbi.nlm.nih.gov/pub/). Exemplary tools include the BLAST system available at http://www.ncbi.nlm.nih.gov, using default settings. Pairwise and ClustalW alignments (BLOSUM30 matrix setting) as well as Kyte-Doolittle hydropathic analysis can be obtained using the MacVector sequence analysis software (Oxford Molecular Group). Watson-Crick complements of the foregoing nucleic acids also are embraced by the invention.

In screening for cancer-associated antigen genes, a Southern blot may be performed using the foregoing conditions, together with a radioactive probe. After washing the membrane to which the DNA is finally transferred, the membrane can be placed against X-ray film to detect the radioactive signal. In screening for the expression of cancer-associated antigen nucleic acids, Northern blot hybridizations using the foregoing conditions can be performed on samples taken from cancer patients or subjects suspected of having a condition characterized by expression of cancer-associated antigen genes. Amplification protocols such as polymerase chain reaction using primers which hybridize to the sequences presented also can be used for detection of the cancer-associated antigen genes or expression thereof.

As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. An isolated nucleic acid as used herein is not a naturally occurring chromosome.

As used herein with respect to polypeptides, “isolated” means separated from its native environment and present in sufficient quantity to permit its identification or use. Isolated, when referring to a protein or polypeptide, means, for example: (i) selectively produced by expression cloning or (ii) purified as by chromatography or electrophoresis. Isolated proteins or polypeptides may, but need not be, substantially pure. The term “substantially pure” means that the proteins or polypeptides are essentially free of other substances with which they may be found in nature or in vivo systems to an extent practical and appropriate for their intended use. Substantially pure polypeptides may be produced by techniques well known in the art. Because an isolated protein may be admixed with a pharmaceutically acceptable carrier in a pharmaceutical preparation, the protein may comprise only a small percentage by weight of the preparation. The protein is nonetheless isolated in that it has been separated from the substances with which it may be associated in living systems, i.e. isolated from other proteins.

The human ramp cancer-associated polypeptide corresponds to SEQ ID NO:12. Encoded polypeptides (e.g., proteins), peptides and antisera thereto are also preferred for diagnosis.

The invention also includes degenerate nucleic acids that include alternative codons to those present in the native materials. For example, serine residues are encoded by the codons TCA, AGT, TCC, TCG, TCT and AGC. Each of the six codons is equivalent for the purposes of encoding a serine residue. Thus, it will be apparent to one of ordinary skill in the art that any of the serine-encoding nucleotide triplets may be employed to direct the protein synthesis apparatus, in vitro or in vivo, to incorporate a serine residue into an elongating cancer-associated antigen polypeptide. Similarly, nucleotide sequence triplets which encode other amino acid residues include, but are not limited to: CCA, CCC, CCG and CCT (proline codons); CGA, CGC, CGG, CGT, AGA and AGG (arginine codons); ACA, ACC, ACG and ACT (threonine codons); AAC and AAT (asparagine codons); and ATA, ATC and ATT (isoleucine codons). Other amino acid residues may be encoded similarly by multiple nucleotide sequences. Thus, the invention embraces degenerate nucleic acids that differ from the biologically isolated nucleic acids in codon sequence due to the degeneracy of the genetic code.

The invention also provides modified nucleic acid molecules which include additions, substitutions and deletions of one or more nucleotides. In preferred embodiments, these modified nucleic acid molecules and/or the polypeptides they encode retain at least one activity or function of the unmodified nucleic acid molecule and/or the polypeptides, such as antigenicity, enzymatic activity, receptor binding, formation of complexes by binding of peptides by MHC class I and class II molecules, etc. In certain embodiments, the modified nucleic acid molecules encode modified polypeptides, preferably polypeptides having conservative amino acid substitutions as are described elsewhere herein. The modified nucleic acid molecules are structurally related to the unmodified nucleic acid molecules and in preferred embodiments are sufficiently structurally related to the unmodified nucleic acid molecules so that the modified and unmodified nucleic acid molecules hybridize under stringent conditions known to one of skill in the art.

For example, modified nucleic acid molecules which encode polypeptides having single amino acid changes can be prepared. Each of these nucleic acid molecules can have one, two or three nucleotide substitutions exclusive of nucleotide changes corresponding to the degeneracy of the genetic code as described herein. Likewise, modified nucleic acid molecules which encode polypeptides having two amino acid changes can be prepared which have, e.g., 2-6 nucleotide changes. Numerous modified nucleic acid molecules like these will be readily envisioned by one of skill in the art, including for example, substitutions of nucleotides in codons encoding amino acids 2 and 3, 2 and 4, 2 and 5, 2 and 6, and so on. In the foregoing example, each combination of two amino acids is included in the set of modified nucleic acid molecules, as well as all nucleotide substitutions which code for the amino acid substitutions. Additional nucleic acid molecules that encode polypeptides having additional substitutions (i.e., 3 or more), additions or deletions (e.g., by introduction of a stop codon or a splice site(s)) also can be prepared and are embraced by the invention as readily envisioned by one of ordinary skill in the art. Any of the foregoing nucleic acids or polypeptides can be tested by routine experimentation for retention of structural relation or activity to the nucleic acids and/or polypeptides disclosed herein.

The invention also provides isolated unique fragments of ramp cancer-associated antigen nucleic acid sequences or complements thereof. A unique fragment is one that is a ‘signature’ for the larger nucleic acid. It, for example, is long enough to assure that its precise sequence is not found in molecules within the human genome outside of the ramp cancer-associated antigen nucleic acids defined above (and human alleles). Those of ordinary skill in the art may apply no more than routine procedures to determine if a fragment is unique within the human genome.

Fragments can be used as probes in Southern and Northern blot assays to identify such nucleic acids, or can be used in amplification assays such as those employing PCR. As known to those skilled in the art, large probes such as 200, 250, 300 or more nucleotides are preferred for certain uses such as Southern and Northern blots, while smaller fragments will be preferred for uses such as PCR. Fragments also can be used to produce fusion proteins for generating antibodies or determining binding of the polypeptide fragments, or for generating immunoassay components. Likewise, fragments can be employed to produce nonfused fragments of the ramp cancer-associated antigen polypeptides, useful, for example, in the preparation of antibodies, and in immunoassays. Fragments further can be used as antisense molecules to inhibit the expression of cancer-associated antigen nucleic acids and polypeptides, particularly for therapeutic purposes as described in greater detail below.

As will be recognized by those skilled in the art, the size of a unique fragment will depend upon its conservancy in the genetic code. Thus, some regions of cancer-associated antigen sequences and complements thereof will require longer segments to be unique while others will require only short segments, typically between 12 and 32 nucleotides (e.g. 12, 13, 14, 15, 16, 17, 18, 19, 20, 21, 22, 23, 24, 25, 26, 27, 28, 29, 30, 31 and 32 or more bases long, up to the entire length of the disclosed sequence. As mentioned above, this disclosure intends to embrace each and every fragment of each sequence, beginning at the first nucleotide, the second nucleotide and so on, up to 8 nucleotides short of the end, and ending anywhere from nucleotide number 8, 9, 10 and so on for each sequence, up to the very last nucleotide (provided the sequence is unique as described above).

Especially preferred include nucleic acids encoding a series of epitopes, known as “polytopes”. The epitopes can be arranged in sequential or overlapping fashion (see, e.g., Thomson et al., Proc. Natl. Acad. Sci. USA 92:5845-5849, 1995; Gilbert et al., Nature Biotechnol. 15:1280-1284, 1997), with or without the natural flanking sequences, and can be separated by unrelated linker sequences if desired. The polytope is processed to generated individual epitopes which are recognized by the immune system for generation of immune responses.

Thus, for example, peptides derived from a ramp polypeptide as disclosed herein, and which are presented by MHC molecules and recognized by CTL or T helper lymphocytes, can be combined with peptides from one or more other cancer-associated antigens (e.g. by preparation of hybrid nucleic acids or polypeptides) to form “polytopes”. The two or more peptides (or nucleic acids encoding the peptides) can be selected from those described herein, or they can include one or more peptides of previously known cancer-associated antigens. Exemplary cancer-associated peptide antigens that can be administered to induce or enhance an immune response are derived from tumor associated genes and encoded proteins including MAGE-A1, MAGE-A2, MAGE-A3, MAGE-A4, MAGE-A5, MAGE-A6, MAGE-A7, MAGE-A8, MAGE-A9, MAGE-A10, MAGE-A 11, MAGE-A12, MAGE-A13, GAGE-1, GAGE-2, GAGE-3, GAGE-4, GAGE-5, GAGE-6, GAGE-7, GAGE-8, BAGE-1, RAGE-1, LB33/MUM-1, PRAME, NAG, MAGE-B2, MAGE-B3, MAGE-B4, tyrosinase, brain glycogen phosphorylase, Melan-A, MAGE-C1, MAGE-C2, NY-ESO-1, LAGE-1, SSX-1, SSX-2 (HOM-MEL-40), SSX-4, SSX-5, SCP-1 and CT-7. See, for example, PCT application publication no. WO96/10577. Other examples will be known to one of ordinary skill in the art (for example, see Coulie, Stem Cells 13:393-403, 1995), and can be used in the invention in a like manner as those disclosed herein. One of ordinary skill in the art can prepare polypeptides comprising one or more peptides and one or more of the foregoing cancer-associated peptides, or nucleic acids encoding such polypeptides, according to standard procedures of molecular biology.

Thus polytopes are groups of two or more potentially immunogenic or immune response stimulating peptides which can be joined together in various arrangements (e.g. concatenated, overlapping). The polytope (or nucleic acid encoding the polytope) can be administered in a standard immunization protocol, e.g. to animals, to test the effectiveness of the polytope in stimulating, enhancing and/or provoking an immune response.

The peptides can be joined together directly or via the use of flanking sequences to form polytopes, and the use of polytopes as vaccines is well known in the art (see, e.g., Thomson et al., Proc. Acad. Natl. Acad. Sci USA 92(13):5845-5849, 1995; Gilbert et al., Nature Biotechnol. 15(12):1280-1284, 1997; Thomson et al., J. Immunol. 157(2):822-826, 1996; Tam et al., J. Exp. Med. 171(1):299-306, 1990). For example, Tam showed that polytopes consisting of both MHC class I and class II binding epitopes successfully generated antibody and protective immunity in a mouse model. Tam also demonstrated that polytopes comprising “strings” of epitopes are processed to yield individual epitopes which are presented by MHC molecules and recognized by CTLs. Thus polytopes containing various numbers and combinations of epitopes can be prepared and tested for recognition by CTLs and for efficacy in increasing an immune response.

It is known that tumors express a set of tumor antigens, of which only certain subsets may be expressed in the tumor of any given patient. Polytopes can be prepared which correspond to the different combination of epitopes representing the subset of tumor rejection antigens expressed in a particular patient. Polytopes also can be prepared to reflect a broader spectrum of tumor rejection antigens known to be expressed by a tumor type. Polytopes can be introduced to a patient in need of such treatment as polypeptide structures, or via the use of nucleic acid delivery systems known in the art (see, e.g., Allsopp et al., Eur. J. Immunol. 26(8):1951-1959, 1996). Adenovirus, pox virus, Ty-virus like particles, adeno-associated virus, plasmids, bacteria, etc. can be used in such delivery. One can test the polytope delivery systems in mouse models to determine efficacy of the delivery system. The systems also can be tested in human clinical trials.

In instances in which a MHC class I molecule presents tumor rejection antigens derived from cancer-associated nucleic acids, the expression vector may also include a nucleic acid sequence coding for the MHC molecule that presents any particular tumor rejection antigen derived from these nucleic acids and polypeptides. Alternatively, the nucleic acid sequence coding for such a MHC molecule can be contained within a separate expression vector. In a situation where the vector contains both coding sequences, the single vector can be used to transfect a cell which does not normally express either one. Where the coding sequences for a cancer-associated antigen precursor and the MHC molecule which presents it are contained on separate expression vectors, the expression vectors can be cotransfected. The ramp cancer-associated antigen precursor coding sequence may be used alone, when, e.g. the host cell already expresses a MHC molecule which presents a cancer-associated antigen derived from precursor molecules. Of course, there is no limit on the particular host cell which can be used. As the vectors which contain the two coding sequences may be used in any antigen-presenting cells if desired, and the gene for cancer-associated antigen precursor can be used in host cells which do not express a MHC molecule which presents a cancer-associated antigen. Further, cell-free transcription systems may be used in lieu of cells.

As mentioned above, the invention embraces antisense oligonucleotides that selectively bind to a nucleic acid molecule encoding a ramp cancer-associated antigen polypeptide, to reduce the expression of ramp cancer-associated antigens. This is desirable in virtually any medical condition wherein a reduction of expression of cancer-associated antigens is desirable, e.g., in the treatment of cancer. This is also useful for in vitro or in vivo testing of the effects of a reduction of expression of one or more cancer-associated antigens.

As used herein, the term “antisense oligonucleotide” or “antisense” describes an oligonucleotide that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and, thereby, inhibits the transcription of that gene and/or the translation of that mRNA. The antisense molecules are designed so as to interfere with transcription or translation of a target gene upon hybridization with the target gene or transcript. Those skilled in the art will recognize that the exact length of the antisense oligonucleotide and its degree of complementarity with its target will depend upon the specific target selected, including the sequence of the target and the particular bases which comprise that sequence. It is preferred that the antisense oligonucleotide be constructed and arranged so as to bind selectively with the target under physiological conditions, i.e., to hybridize substantially more to the target sequence than to any other sequence in the target cell under physiological conditions. Based upon the sequences of nucleic acids encoding cancer-associated antigens, or upon allelic or homologous genomic and/or cDNA sequences, one of skill in the art can easily choose and synthesize any of a number of appropriate antisense molecules for use in accordance with the present invention. In order to be sufficiently selective and potent for inhibition, such antisense oligonucleotides should comprise at least 10 and, more preferably, at least 15 consecutive bases which are complementary to the target, although in certain cases modified oligonucleotides as short as 7 bases in length have been used successfully as antisense oligonucleotides (Wagner et al., Nature Biotechnol. 14:840-844, 1996). Most preferably, the antisense oligonucleotides comprise a complementary sequence of 20-30 bases. Although oligonucleotides may be chosen which are antisense to any region of the gene or mRNA transcripts, in preferred embodiments the antisense oligonucleotides correspond to N-terminal or 5′ upstream sites such as translation initiation, transcription initiation or promoter sites. In addition, 3′-untranslated regions may be targeted. Targeting to mRNA splicing sites has also been used in the art but may be less preferred if alternative mRNA splicing occurs. In addition, the antisense is targeted, preferably, to sites in which mRNA secondary structure is not expected (see, e.g., Sainio et al., Cell Mol. Neurobiol. 14(5):439-457, 1994) and at which proteins are not expected to bind. Finally, although the listed sequences are cDNA sequences, one of ordinary skill in the art may easily derive the genomic DNA corresponding to the cDNA of a cancer-associated antigen. Thus, the present invention also provides for antisense oligonucleotides which are complementary to the genomic DNA corresponding to nucleic acids encoding cancer-associated antigens. Similarly, antisense to allelic cDNAs, homologous cDNAs (e.g., human) and genomic DNAs are enabled without undue experimentation.

In one set of embodiments, the antisense oligonucleotides of the invention may be composed of “natural” deoxyribonucleotides, ribonucleotides, or any combination thereof. That is, the 5′ end of one native nucleotide and the 3′ end of another native nucleotide may be covalently linked, as in natural systems, via a phosphodiester internucleoside linkage. These oligonucleotides may be prepared by art recognized methods which may be carried out manually or by an automated synthesizer. They also may be produced recombinantly by vectors.

In preferred embodiments, however, the antisense oligonucleotides of the invention also may include “modified” oligonucleotides. That is, the oligonucleotides may be modified in a number of ways which do not prevent them from hybridizing to their target but which enhance their stability or targeting or which otherwise enhance their therapeutic effectiveness.

The term “modified oligonucleotide” as used herein describes an oligonucleotide in which (1) at least two of its nucleotides are covalently linked via a synthetic internucleoside linkage (i.e., a linkage other than a phosphodiester linkage between the 5′ end of one nucleotide and the 3′ end of another nucleotide) and/or (2) a chemical group not normally associated with nucleic acids has been covalently attached to the oligonucleotide. Preferred synthetic internucleoside linkages are phosphorothioates, alkylphosphonates, phosphorodithioates, phosphate esters, alkylphosphonothioates, phosphoramidates, carbamates, carbonates, phosphate triesters, acetamidates, carboxymethyl esters and peptides.

The term “modified oligonucleotide” also encompasses oligonucleotides with a covalently modified base and/or sugar. For example, modified oligonucleotides include oligonucleotides having backbone sugars which are covalently attached to low molecular weight organic groups other than a hydroxyl group at the 3′ position and other than a phosphate group at the 5′ position. Thus modified oligonucleotides may include a 2′-O-alkylated ribose group. In addition, modified oligonucleotides may include sugars such as arabinose instead of ribose. The present invention, thus, contemplates pharmaceutical preparations containing modified antisense molecules that are complementary to and hybridizable with, under physiological conditions, nucleic acids encoding cancer-associated antigen polypeptides, together with pharmaceutically acceptable carriers.

As used herein, a “vector” may be any of a number of nucleic acids into which a desired sequence may be inserted by restriction and ligation for transport between different genetic environments or for expression in a host cell. Vectors are typically composed of DNA although RNA vectors are also available. Vectors include, but are not limited to, plasmids, phagemids and virus genomes. A cloning vector is one which is able to replicate autonomously or integrated in the genone in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase. An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., green fluorescent protein). Preferred vectors are those capable of autonomous replication and expression of the structural gene products present in the DNA segments to which they are operably joined.

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript might be translated into the desired protein or polypeptide.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. Especially, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (RNA) encoding a cancer-associated antigen polypeptide or fragment or variant thereof. That heterologous DNA (RNA) is placed under operable control of transcriptional elements to permit the expression of the heterologous DNA in the host cell.

Preferred systems for mRNA expression in mammalian cells are those such as pcDNA3.1 or pRc/CMV (available from Invitrogen, Carlsbad, Calif.) that contain a selectable marker such as a gene that confers G418 resistance (which facilitates the selection of stably transfected cell lines) and the human cytomegalovirus (CMV) enhancer-promoter sequences. Additionally, suitable for expression in primate or canine cell lines is the pCEP4 vector (Invitrogen), which contains an Epstein Barr Virus (EBV) origin of replication, facilitating the maintenance of plasmid as a multicopy extrachromosomal element. Another expression vector is the pEF-BOS plasmid containing the promoter of polypeptide Elongation Factor 1α, which stimulates efficiently transcription in vitro. The plasmid is described by Mishizuma and Nagata (Nuc. Acids Res. 18:5322, 1990), and its use in transfection experiments is disclosed by, for example, Demoulin (Mol. Cell. Biol. 16:4710-4716, 1996). Still another preferred expression vector is an adenovirus, described by Stratford-Perricaudet, which is defective for E1 and E3 proteins (J. Clin. Invest. 90:626-630, 1992). The use of the adenovirus as an Adeno.P1A recombinant for the expression of an antigen is disclosed by Warnier et al., in intradermal injection in mice for immunization against P1A (Int. J. Cancer, 67:303-310, 1996). Additional vectors for delivery of nucleic acid are provided below.

The invention also embraces so-called expression kits, which allow the artisan to prepare a desired expression vector or vectors. Such expression kits include at least separate portions of a vector and one or more of the previously discussed ramp cancer-associated antigen nucleic acid molecules. Other components may be added, as desired, as long as the previously mentioned nucleic acid molecules, which are required, are included. The invention also includes kits for amplification of a ramp cancer-associated antigen nucleic acid, including at least one pair of amplification primers which hybridize to a cancer-associated antigen nucleic acid. The primers preferably are 12-32 nucleotides in length and are non-overlapping to prevent formation of “primer-dimers”. One of the primers will hybridize to one strand of the cancer-associated antigen nucleic acid and the second primer will hybridize to the complementary strand of the cancer-associated antigen nucleic acid, in an arrangement which permits amplification of the cancer-associated antigen nucleic acid. Selection of appropriate primer pairs is standard in the art. For example, the selection can be made with assistance of a computer program designed for such a purpose, optionally followed by testing the primers for amplification specificity and efficiency.

The invention also permits the construction of cancer-associated antigen gene “knock-outs” and transgenics in cells and in animals, providing materials for studying certain aspects of cancer and immune system responses to cancer.

The invention also provides isolated polypeptides (including whole proteins and partial proteins) encoded by the foregoing ramp cancer-associated antigen nucleic acids. Such polypeptides are useful, for example, alone or as fusion proteins to generate antibodies, as components of an immunoassay or diagnostic assay or as therapeutics ramp cancer-associated antigen polypeptides can be isolated from biological samples including tissue or cell homogenates, and can also be expressed recombinantly in a variety of prokaryotic and eukaryotic expression systems by constructing an expression vector appropriate to the expression system, introducing the expression vector into the expression system, and isolating the recombinantly expressed protein. Short polypeptides, including antigenic peptides (such as are presented by MHC molecules on the surface of a cell for immune recognition) also can be synthesized chemically using well-established methods of peptide synthesis.

A unique fragment of a cancer-associated antigen polypeptide, in general, has the features and characteristics of unique fragments as discussed above in connection with nucleic acids. As will be recognized by those skilled in the art, the size of the unique fragment will depend upon factors such as whether the fragment constitutes a portion of a conserved protein domain. Thus, some regions of ramp cancer-associated antigens will require longer segments to be unique while others will require only short segments, typically between 5 and 12 amino acids (e.g. 5, 6, 7, 8, 9, 10, 11 or 12 or more amino acids including each integer up to the full length).

Human homologs of ramp cancer-associated antigen polypeptides are related in sequence to the ramp cancer-associated antigens described herein, and preferably are also related in function. Preferably the homologs are 90% or more identical to one or more portions of the amino acid sequence of the cancer-associated antigens, more preferably are 95% or more identical, and still more preferably are 99% or more identical. Most preferably, the homologs contains at least one fragment of 10 or more amino acids that are identical to the corresponding amino acids of the cancer-associated antigens. In some embodiments a human homolog has the same or similar activity or function as a cancer-associated antigen. Activities and functions include, but are not limited to, enzymatic activity, recognition by antibodies, DNA binding activity, transcriptional activity, binding to MHC molecules, and the like.

Unique fragments and human homologs of a polypeptide preferably are those fragments and homologs which retain a distinct functional capability of the polypeptide. Functional capabilities which can be retained in a unique fragment of a polypeptide include interaction with antibodies, interaction with other polypeptides or fragments thereof, selective binding of nucleic acids or proteins, and enzymatic activity. One important activity is the ability to act as a signature for identifying the polypeptide. Another is the ability to complex with MHC and to provoke in a mammal, preferably a human, an immune response. Those skilled in the art are well versed in methods for selecting unique amino acid sequences, typically on the basis of the ability of the unique fragment to selectively distinguish the sequence of interest from non-family members. A comparison of the sequence of the fragment to those on known databases typically is all that is necessary.

The invention embraces variants of the ramp cancer-associated antigen polypeptides described above. As used herein, a “variant” of a cancer-associated antigen polypeptide is a polypeptide which contains one or more modifications to the primary amino acid sequence of a ramp cancer-associated antigen polypeptide. Modifications which create a cancer-associated antigen variant can be made to a ramp cancer-associated antigen polypeptide 1) to reduce or eliminate an activity of a cancer-associated antigen polypeptide; 2) to enhance a property of a cancer-associated antigen polypeptide, such as protein stability in an expression system or the stability of protein-protein binding; 3) to provide a novel activity or property to a cancer-associated antigen polypeptide, such as addition of an antigenic epitope or addition of a detectable moiety; or 4) to provide equivalent or better binding to a MHC molecule. Modifications to a cancer-associated antigen polypeptide are typically made to the nucleic acid which encodes the cancer-associated antigen polypeptide, and can include deletions, point mutations, truncations, amino acid substitutions and additions of amino acids or non-amino acid moieties. Alternatively, modifications can be made directly to the polypeptide, such as by cleavage, addition of a linker molecule, addition of a detectable moiety, such as biotin, addition of a fatty acid, and the like. Modifications also embrace fusion proteins comprising all or part of the cancer-associated antigen amino acid sequence. One of skill in the art will be familiar with methods for predicting the effect on protein conformation of a change in protein sequence, and can thus “design” a variant cancer-associated antigen polypeptide according to known methods. One example of such a method is described by Dahiyat and Mayo in Science 278:82-87, 1997, whereby proteins can be designed de novo. The method can be applied to a known protein to vary a only a portion of the polypeptide sequence. By applying the computational methods of Dahiyat and Mayo, specific variants of a cancer-associated antigen polypeptide can be proposed and tested to determine whether the variant retains a desired conformation.

In general, variants include ramp cancer-associated antigen polypeptides which are modified specifically to alter a feature of the polypeptide unrelated to its desired physiological activity. For example, cysteine residues can be substituted or deleted to prevent unwanted disulfide linkages. Similarly, certain amino acids can be changed to enhance expression of a cancer-associated antigen polypeptide by eliminating proteolysis by proteases in an expression system (e.g., dibasic amino acid residues in yeast expression systems in which KEX2 protease activity is present).

Mutations of a nucleic acid which encode a ramp cancer-associated antigen polypeptide preferably preserve the amino acid reading frame of the coding sequence, and preferably do not create regions in the nucleic acid which are likely to hybridize to form secondary structures, such a hairpins or loops, which can be deleterious to expression of the variant polypeptide.

Mutations can be made by selecting an amino acid substitution, or by random mutagenesis of a selected site in a nucleic acid which encodes the polypeptide. Variant polypeptides are then expressed and tested for one or more activities to determine which mutation provides a variant polypeptide with the desired properties. Further mutations can be made to variants (or to non-variant cancer-associated antigen polypeptides) which are silent as to the amino acid sequence of the polypeptide, but which provide preferred codons for translation in a particular host. The preferred codons for translation of a nucleic acid in, e.g., E. coli, are well known to those of ordinary skill in the art. Still other mutations can be made to the noncoding sequences of a cancer-associated antigen gene or cDNA clone to enhance expression of the polypeptide. The activity of variants of ramp cancer-associated antigen polypeptides can be tested by cloning the gene encoding the variant cancer-associated antigen polypeptide into a bacterial or mammalian expression vector, introducing the vector into an appropriate host cell, expressing the variant cancer-associated antigen polypeptide, and testing for a functional capability of the ramp cancer-associated antigen polypeptides as disclosed herein. For example, the variant ramp cancer-associated antigen polypeptide can be tested for recognition by CTLs as described in the Examples. Preparation of other variant polypeptides may favor testing of other activities, as will be known to one of ordinary skill in the art.

The skilled artisan will also realize that conservative amino acid substitutions may be made in ramp cancer-associated antigen polypeptides to provide functionally equivalent variants of the foregoing polypeptides, i.e, the variants retain the functional capabilities of the ramp cancer-associated antigen polypeptides. As used herein, a “conservative amino acid substitution” refers to an amino acid substitution which does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Exemplary functionally equivalent variants of the cancer-associated antigen polypeptides include conservative amino acid substitutions of in the amino acid sequences of proteins disclosed herein. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

For example, upon determining that a peptide derived from a cancer-associated antigen polypeptide is presented by an MHC molecule and recognized by CTLs, one can make conservative amino acid substitutions to the amino acid sequence of the peptide, particularly at residues which are thought not to be direct contact points with the MHC molecule. For example, methods for identifying functional variants of HLA class II binding peptides are provided in a published PCT application of Strominger and Wucherpfennig (PCT/US96/03182). Peptides bearing one or more amino acid substitutions also can be tested for concordance with known HLA/MHC motifs prior to synthesis using, e.g. the computer program described by D'Amaro and Drijfhout (D'Amaro et al., Human Immunol. 43:13-18, 1995; Drijfhout et al., Human Immunol. 43:1-12, 1995). The substituted peptides can then be tested for binding to the MHC molecule and recognition by CTLs when bound to MHC. These variants can be tested for improved stability and are useful, inter alia, in vaccine compositions.

Conservative amino-acid substitutions in the amino acid sequence of cancer-associated antigen polypeptides to produce functionally equivalent variants of cancer-associated antigen polypeptides typically are made by alteration of a nucleic acid encoding a cancer-associated antigen polypeptide. Such substitutions can be made by a variety of methods known to one of ordinary skill in the art. For example, amino acid substitutions may be made by PCR-directed mutation, site-directed mutagenesis according to the method of Kunkel (Kunkel, Proc. Nat. Acad. Sci. U.S.A. 82: 488-492, 1985), or by chemical synthesis of a gene encoding a cancer-associated antigen polypeptide. Where amino acid substitutions are made to a small unique fragment of a cancer-associated antigen polypeptide, such as an antigenic epitope recognized by cytolytic T lymphocytes, the substitutions can be made by directly synthesizing the peptide. The activity of functionally equivalent fragments of cancer-associated antigen polypeptides can be tested by cloning the gene encoding the altered cancer-associated antigen polypeptide into a bacterial or mammalian expression vector, introducing the vector into an appropriate host cell, expressing the altered cancer-associated antigen polypeptide, and testing for a functional capability of the cancer-associated antigen polypeptides as disclosed herein. Peptides which are chemically synthesized can be tested directly for function.

The invention as described herein has a number of uses, some of which are described elsewhere herein. First, the invention permits isolation of the ramp cancer-associated antigen protein molecules. A variety of methodologies well-known to the skilled practitioner can be utilized to obtain isolated cancer-associated antigen molecules. The polypeptide may be purified from cells which naturally produce the polypeptide by chromatographic means or immunological recognition. Alternatively, an expression vector may be introduced into cells to cause production of the polypeptide. In another method, mRNA transcripts may be microinjected or otherwise introduced into cells to cause production of the encoded polypeptide. Translation of mRNA in cell-free extracts such as the reticulocyte lysate system also may be used to produce polypeptide. Those skilled in the art also can readily follow known methods for isolating cancer-associated antigen polypeptides. These include, but are not limited to, immunochromatography, HPLC, size-exclusion chromatography, ion-exchange chromatography and immune-affinity chromatography.

The isolation and identification of the ramp cancer-associated antigen gene also makes it possible for the artisan to diagnose a disorder characterized by expression of ramp cancer-associated antigens. These methods involve determining expression of ramp nucleic acids, and/or encoded ramp polypeptides and/or peptides derived therefrom. In the former situation, such determinations can be carried out via any standard nucleic acid determination assay, including the polymerase chain reaction.

The invention also makes it possible isolate proteins which bind to ramp cancer-associated antigens as disclosed herein, including antibodies and cellular binding partners of the ramp cancer-associated antigens. Additional uses are described further herein.

The invention also provides, in certain embodiments, “dominant negative” polypeptides derived from ramp polypeptides. A dominant negative polypeptide is an inactive variant of a protein, which, by interacting with the cellular machinery, displaces an active protein from its interaction with the cellular machinery or competes with the active protein, thereby reducing the effect of the active protein. For example, a dominant negative receptor which binds a ligand but does not transmit a signal in response to binding of the ligand can reduce the biological effect of expression of the ligand. Likewise, a dominant negative catalytically-inactive kinase which interacts normally with target proteins but does not phosphorylate the target proteins can reduce phosphorylation of the target proteins in response to a cellular signal. Similarly, a dominant negative transcription factor which binds to a promoter site in the control region of a gene but does not increase gene transcription can reduce the effect of a normal transcription factor by occupying promoter binding sites without increasing transcription.

The end result of the expression of a dominant negative polypeptide in a cell is a reduction in function of active proteins. One of ordinary skill in the art can assess the potential for a dominant negative variant of a protein, and using standard mutagenesis techniques to create one or more dominant negative variant polypeptides. For example, given the teachings contained herein of cancer-associated antigens, one of ordinary skill in the art can modify the sequence of the ramp cancer-associated antigen by site-specific mutagenesis, scanning mutagenesis, partial gene deletion or truncation, and the like. See, e.g., U.S. Pat. No. 5,580,723 and Sambrook et al., Molecular Cloning: A Laboratory Manual, Second Edition, Cold Spring Harbor Laboratory Press, 1989. The skilled artisan then can test the population of mutagenized polypeptides for diminution in a selected and/or for retention of such an activity. Other similar methods for creating and testing dominant negative variants of a protein will be apparent to one of ordinary skill in the art.

The invention also involves agents such as polypeptides which bind to ramp polypeptides. Such binding agents can be used, for example, in screening assays to detect the presence or absence of ramp polypeptides and complexes of ramp polypeptides and their binding partners and in purification protocols to isolate ramp polypeptides and complexes of ramp polypeptides and their binding partners. Such agents also can be used to inhibit the native activity of the ramp polypeptides, for example, by binding to such polypeptides.

The invention, therefore, embraces peptide binding agents which, for example, can be antibodies or fragments of antibodies having the ability to selectively bind to ramp polypeptides. Antibodies include polyclonal and monoclonal antibodies, prepared according to conventional methodology.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well-established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. See, e.g., U.S. Pat. Nos. 4,816,567, 5,225,539, 5,585,089, 5,693,762 and 5,859,205.

Thus, for example, PCT International Publication Number WO 92/04381 teaches the production and use of humanized murine RSV antibodies in which at least a portion of the murine FR regions have been replaced by FR regions of human origin. Such antibodies, including fragments of intact antibodies with antigen-binding ability, are often referred to as “chimeric” antibodies.

Thus, as will be apparent to one of ordinary skill in the art, the present invention also provides for F(ab′)₂, Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)₂ fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

Thus, the invention involves polypeptides of numerous size and type that bind specifically to ramp polypeptides, and complexes of both ramp polypeptides and their binding partners. These polypeptides may be derived also from sources other than antibody technology. For example, such polypeptide binding agents can be provided by degenerate peptide libraries which can be readily prepared in solution, in immobilized form or as phage display libraries. Combinatorial libraries also can be synthesized of peptides containing one or more amino acids. Libraries further can be synthesized of peptoids and non-peptide synthetic moieties.

Phage display can be particularly effective in identifying binding peptides useful according to the invention. Briefly, one prepares a phage library (using e.g. m13, fd, or lambda phage), displaying inserts from 4 to about 80 amino acid residues using conventional procedures. The inserts may represent, for example, a completely degenerate or biased array. One then can select phage-bearing inserts which bind to the ramp cancer-associated antigen polypeptide. This process can be repeated through several cycles of reselection of phage that bind to the cancer-associated antigen polypeptide. Repeated rounds lead to enrichment of phage bearing particular sequences. DNA sequence analysis can be conducted to identify the sequences of the expressed polypeptides. The minimal linear portion of the sequence that binds to the cancer-associated antigen polypeptide can be determined. One can repeat the procedure using a biased library containing inserts containing part or all of the minimal linear portion plus one or more additional degenerate residues upstream or downstream thereof. Yeast two-hybrid screening methods also may be used to identify polypeptides that bind to the ramp polypeptides. Thus, the ramp polypeptides of the invention, or a fragment thereof, can be used to screen peptide libraries, including phage display libraries, to identify and select peptide binding partners of the ramp polypeptides of the invention. Such molecules can be used, as described, for screening assays, for purification protocols, for interfering directly with the functioning of ramp and for other purposes that will be apparent to those of ordinary skill in the art.

As detailed herein, the foregoing antibodies and other binding molecules may be used for example to identify tissues expressing protein or to purify protein. Antibodies also may be coupled to specific diagnostic labeling agents for imaging of cells and tissues that express ramp or to therapeutically useful agents according to standard coupling procedures. Diagnostic agents include, but are not limited to, barium sulfate, iocetamic acid, iopanoic acid, ipodate calcium, diatrizoate sodium, diatrizoate meglumine, metrizamide, tyropanoate sodium and radiodiagnostics including positron emitters such as fluorine-18 and carbon-11, gamma emitters such as iodine-123, technitium-99m, iodine-131 and indium-111, nuclides for nuclear magnetic resonance such as fluorine and gadolinium. Other diagnostic agents useful in the invention will be apparent to one of ordinary skill in the art. As used herein, “therapeutically useful agents” include any therapeutic molecule which desirably is targeted selectively to a cell expressing ramp, including antineoplastic agents, radioiodinated compounds, toxins, other cytostatic or cytolytic drugs, and so forth. Antineoplastic therapeutics are well known and include: aminoglutethimide, azathioprine, bleomycin sulfate, busulfan, carmustine, chlorambucil, cisplatin, cyclophosphamide, cyclosporine, cytarabidine, dacarbazine, dactinomycin, daunorubicin, doxorubicin, taxol, etoposide, fluorouracil, interferon-α, lomustine, mercaptopurine, methotrexate, mitotane, procarbazine HCl, thioguanine, vinblastine sulfate and vincristine sulfate. Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division). Toxins can be proteins such as, for example, pokeweed anti-viral protein, cholera toxin, pertussis toxin, ricin, gelonin, abrin, diphtheria exotoxin, or Pseudomonas exotoxin. Toxin moieties can also be high energy-emitting radionuclides such as cobalt-60.

In the foregoing methods, antibodies prepared according to the invention also preferably are specific for the cancer-associated antigen/MHC complexes described herein.

When “disorder” is used herein, it refers to any pathological condition where the cancer-associated antigens are expressed. An example of such a disorder is cancer. For human cancers, particular examples include, but are not limited to: bladder cancer, breast cancer, lung cancer, colon cancer, fibrosarcoma, and hepatoma.

Samples of tissue and/or cells for use in the various methods described herein can be obtained through standard methods such as tissue biopsy, including punch biopsy and cell scraping, and collection of blood or other bodily fluids by aspiration or other methods.

In certain embodiments of the invention, an immunoreactive cell sample is removed from a subject. By “immunoreactive cell” is meant a cell which can mature into an immune cell (such as a B cell, a helper T cell, or a cytolytic T cell) upon appropriate stimulation. Thus immunoreactive cells include CD34⁺ hematopoietic stem cells, immature T cells and immature B cells. When it is desired to produce cytolytic T cells which recognize a cancer-associated antigen, the immunoreactive cell is contacted with a cell which expresses a cancer-associated antigen under conditions favoring production, differentiation and/or selection of cytolytic T cells; the differentiation of the T cell precursor into a cytolytic T cell upon exposure to antigen is similar to clonal selection of the immune system.

Some therapeutic approaches based upon the disclosure are premised on a response by a subject's immune system, leading to lysis of antigen presenting cells, such as cancer cells which present one or more cancer-associated antigens. One such approach is the administration of autologous CTLs specific to a cancer-associated antigen/MHC complex to a subject with abnormal cells of the phenotype at issue. It is within the ability of one of ordinary skill in the art to develop such CTLs in vitro. An example of a method for T cell differentiation is presented in International Application number PCT/US96/05607. Generally, a sample of cells taken from a subject, such as blood cells, are contacted with a cell presenting the complex and capable of provoking CTLs to proliferate. The target cell can be a transfectant, such as a COS cell. These transfectants present the desired complex of their surface and, when combined with a CTL of interest, stimulate its proliferation. COS cells are widely available, as are other suitable host cells. Specific production of CTL clones is well known in the art. The clonally expanded autologous CTLs then are administered to the subject.

Another method for selecting antigen-specific CTL clones has recently been described (Altman et al., Science 274:94-96, 1996; Dunbar et al., Curr. Biol. 8:413-416, 1998), which is described above herein. The reactive CTLs isolated by the method can then be expanded in vitro for use as described herein.

To detail a therapeutic methodology, referred to as adoptive transfer (Greenberg, J. Immunol. 136(5): 1917, 1986; Riddel et al., Science 257: 238, 1992; Lynch et al, Eur. J. Immunol. 21: 1403-1410,1991; Kast et al., Cell 59: 603-614, 1989), cells presenting the desired complex (e.g., dendritic cells) are combined with CTLs leading to proliferation of the CTLs specific thereto. The proliferated CTLs are then administered to a subject with a cellular abnormality which is characterized by certain of the abnormal cells presenting the particular complex. The CTLs then lyse the abnormal cells, thereby achieving the desired therapeutic goal.

The foregoing therapy assumes that at least some of the subject's abnormal cells present the relevant HLA/cancer-associated antigen complex. This can be determined very easily, as the art is very familiar with methods for identifying cells which present a particular HLA molecule, as well as how to identify cells expressing DNA of the pertinent sequences, in this case, a cancer-associated antigen sequence. Once cells presenting the relevant complex are identified via the foregoing screening methodology, they can be combined with a sample from a patient, where the sample contains CTLs. If the complex presenting cells are lysed by the mixed CTL sample, then it can be assumed that a cancer-associated antigen is being presented, and the subject is an appropriate candidate for the therapeutic approaches set forth supra.

Adoptive transfer is not the only form of therapy that is available in accordance with the invention. CTLs can also be provoked in vivo, using a number of approaches. One approach is the use of non-proliferative cells expressing the complex. The cells used in this approach may be those that normally express the complex, such as irradiated tumor cells or cells transfected with one or both of the genes necessary for presentation of the complex (i.e. the antigenic peptide and the presenting MHC molecule). Chen et al. (Proc. Natl. Acad. Sci. USA 88: 110-114,1991) exemplifies this approach, showing the use of transfected cells expressing HPV E7 peptides in a therapeutic regime. Various cell types may be used. Similarly, vectors carrying one or both of the genes of interest may be used. Viral or bacterial vectors are especially preferred. For example, nucleic acids which encode a cancer-associated antigen polypeptide or peptide may be operably linked to promoter and enhancer sequences which direct expression of the cancer-associated antigen polypeptide or peptide in certain tissues or cell types. The nucleic acid may be incorporated into an expression vector. Expression vectors may be unmodified extrachromosomal nucleic acids, plasmids or viral genomes constructed or modified to enable insertion of exogenous nucleic acids, such as those encoding cancer-associated antigen, as described elsewhere herein. Nucleic acids encoding a cancer-associated antigen also may be inserted into a retroviral genome, thereby facilitating integration of the nucleic acid into the genome of the target tissue or cell type. In these systems, the gene of interest is carried by a microorganism, e.g., a Vaccinia virus, pox virus, herpes simplex virus, retrovirus or adenovirus, and the materials de facto “infect” host cells. The cells which result present the complex of interest, and are recognized by autologous CTLs, which then proliferate.

A similar effect can be achieved by combining the cancer-associated antigen or a stimulatory fragment thereof with an adjuvant to facilitate incorporation into antigen presenting cells in vivo. The cancer-associated antigen polypeptide is processed to yield the peptide partner of the MHC molecule while a cancer-associated antigen peptide may be presented without the need for further processing. Generally, subjects can receive an intradermal injection of an effective amount of the cancer-associated antigen. Initial doses can be followed by booster doses, following immunization protocols standard in the art.

The invention involves the use of various materials disclosed herein to “immunize” subjects or as “vaccines”. As used herein, “immunization” or “vaccination” means increasing or activating an immune response against an antigen. It does not require elimination or eradication of a condition but rather contemplates the clinically favorable enhancement of an immune response toward an antigen. Generally accepted animal models, including the MethA fibroasrcoma model used herein, can be used for testing of immunization against cancer using a cancer-associated antigen nucleic acid. For example, human cancer cells can be introduced into a mouse to create a tumor, and one or more cancer-associated antigen nucleic acids can be delivered by the methods described herein. The effect on the cancer cells (e.g., reduction of tumor size) can be assessed as a measure of the effectiveness of the cancer-associated antigen nucleic acid immunization. Of course, testing of the foregoing animal model using more conventional methods for immunization include the administration of one or more cancer-associated antigen polypeptides or peptides derived therefrom, optionally combined with one or more adjuvants and/or cytokines to boost the immune response. Methods for immunization, including formulation of a vaccine composition and selection of doses, route of administration and the schedule of administration (e.g. primary and one or more booster doses), are well known in the art. The tests also can be performed in humans, where the end point is to test for the presence of enhanced levels of circulating CTLs against cells bearing the antigen, to test for levels of circulating antibodies against the antigen, to test for the presence of cells expressing the antigen and so forth.

As part of the immunization compositions, one or more cancer-associated antigens or stimulatory fragments thereof are administered with one or more adjuvants to induce an immune response or to increase an immune response. An adjuvant is a substance incorporated into or administered with antigen which potentiates the immune response. Adjuvants may enhance the immunological response by providing a reservoir of antigen (extracellularly or within macrophages), activating macrophages and stimulating specific sets of lymphocytes. Adjuvants of many kinds are well known in the art. Specific examples of adjuvants include monophosphoryl lipid A (MPL, SmithKline Beecham), a congener obtained after purification and acid hydrolysis of Salmonella minnesota Re 595 lipopolysaccharide; saponins including QS21 (SmithKline Beecham), a pure QA-21 saponin purified from Quillja saponaria extract; DQS21, described in PCT application WO96/33739 (SmithKline Beecham); QS-7, QS-17, QS-18, and QS-L1 (So et al., Mol. Cells 7:178-186, 1997); incomplete Freund's adjuvant; complete Freund's adjuvant; montanide; alum; CpG oligonucleotides (see e.g. Kreig et al., Nature 374:546-9, 1995); and various water-in-oil emulsions prepared from biodegradable oils such as squalene and/or tocopherol. Preferably, the peptides are administered mixed with a combination of DQS21/MPL. The ratio of DQS21 to MPL typically will be about 1:10 to 10:1, preferably about 1:5 to 5:1 and more preferably about 1:1. Typically for human administration, DQS21 and MPL will be present in a vaccine formulation in the range of about 1 μg to about 100 μg. Other adjuvants are known in the art and can be used in the invention (see, e.g. Goding, Monoclonal Antibodies: Principles and Practice, 2nd Ed., 1986). Methods for the preparation of mixtures or emulsions of peptide and adjuvant are well known to those of skill in the art of vaccination.

Other agents which stimulate the immune response of the subject can also be administered to the subject. For example, other cytokines are also useful in vaccination protocols as a result of their lymphocyte regulatory properties. Many other cytokines useful for such purposes will be known to one of ordinary skill in the art, including interleukin-12 (IL-12) which has been shown to enhance the protective effects of vaccines (see, e.g., Science 268: 1432-1434, 1995), GM-CSF and IL-18. Thus cytokines can be administered in conjunction with antigens and adjuvants to increase the immune response to the antigens.

There are a number of immune response potentiating compounds that can be used in vaccination protocols. These include costimulatory molecules provided in either protein or nucleic acid form. Such costimulatory molecules include the B7-1 and B7-2 (CD80 and CD86 respectively) molecules which are expressed on dendritic cells (DC) and interact with the CD28 molecule expressed on the T cell. This interaction provides costimulation (signal 2) to an antigen/MHC/TCR stimulated (signal 1) T cell, increasing T cell proliferation and effector function. B7 also interacts with CTLA4 (CD152) on T cells and studies involving CTLA4 and B7 ligands indicate that the B7-CTLA4 interaction can enhance antitumor immunity and CTL proliferation (Zheng P., et al. Proc. Natl. Acad. Sci. USA 95 (11):6284-6289 (1998)).

B7 typically is not expressed on tumor cells so they are not efficient antigen presenting cells (APCs) for T cells. Induction of B7 expression would enable the tumor cells to stimulate more efficiently CTL proliferation and effector function. A combination of B7/IL-6/IL-12 costimulation has been shown to induce IFN-gamma and a Th1 cytokine profile in the T cell population leading to further enhanced T cell activity (Gajewski et al., J. Immunol, 154:5637-5648 (1995)). Tumor cell transfection with B7 has ben discussed in relation to in vitro CTL expansion for adoptive transfer immunotherapy by Wang et al., (J. Immunol., 19:1-8 (1986)). Other delivery mechanisms for the B7 molecule would include nucleic acid (naked DNA) immunization (Kim J., et al. Nat. Biotechnol., 15:7:641-646 (997)) and recombinant viruses such as adeno and pox (Wendtner et al., Gene Ther., 4:7:726-735 (1997)). These systems are all amenable to the construction and use of expression cassettes for the coexpression of B7 with other molecules of choice such as the antigens or fragment(s) of antigens discussed herein (including polytopes) or cytokines. These delivery systems can be used for induction of the appropriate molecules in vitro and for in vivo vaccination situations. The use of anti-CD28 antibodies to directly stimulate T cells in vitro and in vivo could also be considered. Similarly, the inducible co-stimulatory molecule ICOS which induces T cell responses to foreign antigen could be modulated, for example, by use of anti-ICOS antibodies (Hutloff et al., Nature 397:263-266, 1999).

Lymphocyte function associated antigen-3 (LFA-3) is expressed on APCs and some tumor cells and interacts with CD2 expressed on T cells. This interaction induces T cell IL-2 and IFN-gamma production and can thus complement but not substitute, the B7/CD28 costimulatory interaction (Parra et al., J. Immunol., 158:637-642 (1997), Fenton et al., J. Immunother., 21:2:95-108 (1998)).

Lymphocyte function associated antigen-1 (LFA-1) is expressed on leukocytes and interacts with ICAM-1 expressed on APCs and some tumor cells. This interaction induces T cell IL-2 and IFN-gamma production and can thus complement but not substitute, the B7/CD28 costimulatory interaction (Fenton et al., J. Immunother., 21:2:95-108 (1998)). LFA-1 is thus a further example of a costimulatory molecule that could be provided in a vaccination protocol in the various ways discussed above for B7.

Complete CTL activation and effector function requires Th cell help through the interaction between the Th cell CD40L (CD40 ligand) molecule and the CD40 molecule expressed by DCs (Ridge et al., Nature, 393:474 (1998), Bennett et al., Nature, 393:478 (1998), Schoenberger et al., Nature, 393:480 (1998)). This mechanism of this costimulatory signal is likely to involve upregulation of B7 and associated IL-6/IL-12 production by the DC (APC). The CD40-CD40L interaction thus complements the signal 1 (antigen/MHC-TCR) and signal 2 (B7-CD28) interactions.

The use of anti-CD40 antibodies to stimulate DC cells directly, would be expected to enhance a response to tumor antigens which are normally encountered outside of a inflammatory context or are presented by non-professional APCs (tumor cells). In these situations Th help and B7 costimulation signals are not provided. This mechanism might be used in the context of antigen pulsed DC based therapies or in situations where Th epitopes have not been defined within known TRA precursors.

A ramp polypeptide, or a fragment thereof, also can be used to isolate its native binding partners. Isolation of such binding partners may be performed according to well-known methods. For example, isolated ramp polypeptides can be attached to a substrate (e.g., chromatographic media, such as polystyrene beads, or a filter), and then a solution suspected of containing the binding partner may be applied to the substrate. If a binding partner which can interact with ramp polypeptides is present in the solution, then it will bind to the substrate-bound ramp polypeptide. The binding partner then may be isolated.

It will also be recognized that the invention embraces the use of the ramp cDNA sequences in expression vectors, as well as to transfect host cells and cell lines, be these prokaryotic (e.g., E. coli), or eukaryotic (e.g., dendritic cells, B cells, CHO cells, COS cells, yeast expression systems and recombinant baculovirus expression in insect cells). Especially useful are mammalian cells such as human, mouse, hamster, pig, goat, primate, etc. They may be of a wide variety of tissue types, and include primary cells and cell lines. Specific examples include dendritic cells, peripheral blood leukocytes, bone marrow stem cells and embryonic stem cells. The expression vectors require that the pertinent sequence, i.e., those nucleic acids described supra, be operably linked to a promoter.

The invention also contemplates delivery of nucleic acids, polypeptides or peptides for vaccination. Delivery of polypeptides and peptides can be accomplished according to standard vaccination protocols which are well known in the art. In another embodiment, the delivery of nucleic acid is accomplished by ex vivo methods, i.e. by removing a cell from a subject, genetically engineering the cell to include a cancer-associated antigen, and reintroducing the engineered cell into the subject. One example of such a procedure is outlined in U.S. Pat. No. 5,399,346 and in exhibits submitted in the file history of that patent, all of which are publicly available documents. In general, it involves introduction in vitro of a functional copy of a gene into a cell(s) of a subject, and returning the genetically engineered cell(s) to the subject. The functional copy of the gene is under operable control of regulatory elements which permit expression of the gene in the genetically engineered cell(s). Numerous transfection and transduction techniques as well as appropriate expression vectors are well known to those of ordinary skill in the art, some of which are described in PCT application WO95/00654. In vivo nucleic acid delivery using vectors such as viruses and targeted liposomes also is contemplated according to the invention.

In preferred embodiments, a virus vector for delivering a nucleic acid encoding a cancer-associated antigen is selected from the group consisting of adenoviruses, adeno-associated viruses, poxviruses including vaccinia viruses and attenuated poxviruses, Semliki Forest virus, Venezuelan equine encephalitis virus, retroviruses, Sindbis virus, and Ty virus-like particle. Examples of viruses and virus-like particles which have been used to deliver exogenous nucleic acids include: replication-defective adenoviruses (e.g., Xiang et al., Virology 219:220-227, 1996; Eloit et al., J. Virol. 7:5375-5381, 1997; Chengalvala et al., Vaccine 15:335-339, 1997), a modified retrovirus (Townsend et al., J. Virol. 71:3365-3374, 1997), a nonreplicating retrovirus (Irwin et al., J. Virol. 68:5036-5044, 1994), a replication defective Semliki Forest virus (Zhao et al., Proc. Natl. Acad. Sci. USA 92:3009-3013, 1995), canarypox virus and highly attenuated vaccinia virus derivative (Paoletti, Proc. Natl. Acad. Sci. USA 93:11349-11353, 1996), non-replicative vaccinia virus (Moss, Proc. Natl. Acad. Sci. USA 93:11341-11348, 1996), replicative vaccinia virus (Moss, Dev. Biol. Stand. 82:55-63, 1994), Venzuelan equine encephalitis virus (Davis et al., J. Virol. 70:3781-3787, 1996), Sindbis virus (Pugachev et al., Virology 212:587-594, 1995), and Ty virus-like particle (Allsopp et al., Eur. J. Immunol 26:1951-1959, 1996). In preferred embodiments, the virus vector is an adenovirus.

Another preferred virus for certain applications is the adeno-associated virus, a double-stranded DNA virus. The adeno-associated virus is capable of infecting a wide range of cell types and species and can be engineered to be replication-deficient. It further has advantages, such as heat and lipid solvent stability, high transduction frequencies in cells of diverse lineages, including hematopoietic cells, and lack of superinfection inhibition thus allowing multiple series of transductions. The adeno-associated virus can integrate into human cellular DNA in a site-specific manner, thereby minimizing the possibility of insertional mutagenesis and variability of inserted gene expression. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

In general, other preferred viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA. Adenoviruses and retroviruses have been approved for human gene therapy trials. In general, the retroviruses are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, M., “Gene Transfer and Expression, A Laboratory Manual,” W. H. Freeman Co., New York (1990) and Murry, E. J. Ed. “Methods in Molecular Biology,” vol. 7, Humana Press, Inc., Clifton, N.J. (1991).

Preferably the foregoing nucleic acid delivery vectors: (1) contain exogenous genetic material that can be transcribed and translated in a mammalian cell and that can induce an immune response in a host, and (2) contain on a surface a ligand that selectively binds to a receptor on the surface of a target cell, such as a mammalian cell, and thereby gains entry to the target cell.

Various techniques may be employed for introducing nucleic acids of the invention into cells, depending on whether the nucleic acids are introduced in vitro or in vivo in a host. Such techniques include transfection of nucleic acid-CaPO₄ precipitates, transfection of nucleic acids associated with DEAE, transfection or infection with the foregoing viruses including the nucleic acid of interest, liposome mediated transfection, and the like. For certain uses, it is preferred to target the nucleic acid to particular cells. In such instances, a vehicle used for delivering a nucleic acid of the invention into a cell (e.g., a retrovirus, or other virus; a liposome) can have a targeting molecule attached thereto. For example, a molecule such as an antibody specific for a surface membrane protein on the target cell or a ligand for a receptor on the target cell can be bound to or incorporated within the nucleic acid delivery vehicle. Preferred antibodies include antibodies which selectively bind a ramp polypeptide, alone or as a complex with a MHC molecule. Especially preferred are monoclonal antibodies. Where liposomes are employed to deliver the nucleic acids of the invention, proteins which bind to a surface membrane protein associated with endocytosis may be incorporated into the liposome formulation for targeting and/or to facilitate uptake. Such proteins include capsid proteins or fragments thereof tropic for a particular cell type, antibodies for proteins which undergo internalization in cycling, proteins that target intracellular localization and enhance intracellular half life, and the like. Polymeric delivery systems also have been used successfully to deliver nucleic acids into cells, as is known by those skilled in the art. Such systems even permit oral delivery of nucleic acids.

When administered, the therapeutic compositions of the present invention can be administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines and optionally other therapeutic agents.

The therapeutics of the invention can be administered by any conventional route, including injection or by gradual infusion over time. The administration may, for example, be oral, intravenous, intraperitoneal, intramuscular, intracavity, subcutaneous, or transdermal. When antibodies are used therapeutically, a preferred route of administration is by pulmonary aerosol. Techniques for preparing aerosol delivery systems containing antibodies are well known to those of skill in the art. Generally, such systems should utilize components which will not significantly impair the biological properties of the antibodies, such as the paratope binding capacity (see, for example, Sciarra and Cutie, “Aerosols,” in Remington's Pharmaceutical Sciences, 18th edition, 1990, pp 1694-1712; incorporated by reference). Those of skill in the art can readily determine the various parameters and conditions for producing antibody aerosols without resort to undue experimentation. When using antisense preparations of the invention, slow intravenous administration is preferred.

The compositions of the invention are administered in effective amounts. An “effective amount” is that amount of a cancer-associated antigen composition that alone, or together with further doses, produces the desired response, e.g. increases an immune response to the cancer-associated antigen. In the case of treating a particular disease or condition characterized by expression of one or more cancer-associated antigens, such as cancer, the desired response is inhibiting the progression of the disease. This may involve only slowing the progression of the disease temporarily, although more preferably, it involves halting the progression of the disease permanently. This can be monitored by routine methods or can be monitored according to diagnostic methods of the invention discussed herein. The desired response to treatment of the disease or condition also can be delaying the onset or even preventing the onset of the disease or condition.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The pharmaceutical compositions used in the foregoing methods preferably are sterile and contain an effective amount of cancer-associated antigen or nucleic acid encoding cancer-associated antigen for producing the desired response in a unit of weight or volume suitable for administration to a patient. The response can, for example, be measured by determining the immune response following administration of the cancer-associated antigen composition via a reporter system by measuring downstream effects such as gene expression, or by measuring the physiological effects of the cancer-associated antigen composition, such as regression of a tumor or decrease of disease symptoms. Other assays will be known to one of ordinary skill in the art and can be employed for measuring the level of the response.

The doses of ramp compositions (e.g., polypeptide, peptide, antibody, cell or nucleic acid) administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

In general, for treatments for eliciting or increasing an immune response, doses of cancer-associated antigen are formulated and administered in doses between 1 ng and 1 mg, and preferably between 10 ng and 100 μg, according to any standard procedure in the art. Where nucleic acids encoding cancer-associated antigen of variants thereof are employed, doses of between 1 ng and 0.1 mg generally will be formulated and administered according to standard procedures. Other protocols for the administration of cancer-associated antigen compositions will be known to one of ordinary skill in the art, in which the dose amount, schedule of injections, sites of injections, mode of administration (e.g., intra-tumoral) and the like vary from the foregoing. Administration of ramp compositions to mammals other than humans, e.g. for testing purposes or veterinary therapeutic purposes, is carried out under substantially the same conditions as described above.

Where cancer-associated antigen peptides are used for vaccination, modes of administration which effectively deliver the cancer-associated antigen and adjuvant, such that an immune response to the antigen is increased, can be used. For administration of a cancer-associated antigen peptide in adjuvant, preferred methods include intradermal, intravenous, intramuscular and subcutaneous administration. Although these are preferred embodiments, the invention is not limited by the particular modes of administration disclosed herein. Standard references in the art (e.g., Remington's Pharmaceutical Sciences, 18th edition, 1990) provide modes of administration and formulations for delivery of immunogens with adjuvant or in a non-adjuvant carrier.

When administered, the pharmaceutical preparations of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

A ramp composition may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as a syrup, elixir or an emulsion.

Compositions suitable for parenteral administration conveniently comprise a sterile aqueous or non-aqueous preparation of cancer-associated antigen polypeptides or nucleic acids, which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono-or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

### EXAMPLES

Materials and Methods for Examples 1-4

Tumors and cell lines. RL♂1 and RL♀8 are radiation-induced leukemias in BALB/c mice (Nakayama, et al., Proc. Natl. Acad. Sci. USA 76:3486-3490, 1979). RVC is a leukemia induced by injection of radiation-leukemia virus (RadLV) into a neonatal BALB/c mouse (Stockert, et al., J. Exp. Med. 149:200-215, 1979; Nakayama et al., Cancer Res. 44:5138-5144, 1984). Meth A and CMS8 are methylcholanthrene-induced sarcomas in BALB/c mice (DeLeo et al., J. Exp. Med. 146:720-734, 1977; Palladino et al., Cancer Res. 47:5704-5709, 1987). Meth A(p) is the parental Meth A line. Meth A (sv) is a variant line which became sensitive to lysis by cytotoxic T-lymphocytes and was provided by Dr. H. Shiku (Mie University School of Medicine, Mie, Japan). MOPC-70A is a mineral oil-induced myeloma in a BALB/c mouse (Potter, 1967, “The plasma cell tumors and myeloma protein of mice” In: Methods in Cancer Research. H. Busch, Ed. Academic Press, New York, 106-157). P1.HTR (Wolfel et al., Immunogenetics 26:178-187, 1987) is a subline of P815 which is a methylcholanthrene-induced mastocytoma in a DBA/2 mouse (Dunn and Potter, J. Natl. Cancer Inst. 18:587-601, 1957).

Antibodies. Anti-L3T4 (CD4) mAb, a rat antibody of the IgG2b immunoglobulin class, produced by hybridoma GK1.5 (Dialynas et al., J. Immunol. 131:2445-2451, 1983), was provided by Dr. F. Fitch (University of Chicago, Chicago, Ill.). Anti-Lyt-2.2 (CD8) mAb, a mouse antibody of the IgG2a class, produced by hybridoma 19/178 was provided by Dr. G. Hammerling (Memorial Sloan-Kettering Cancer Center, New York). Anti-H-2K^{d} and anti-H-2D^{d} are mouse antibodies produced by hybridomas HB159 and HB102 respectively. Anti-H-2L² mAb is a mouse IgG2a antibody produced by hybridoma 30-5-7 (Ozato et al., J. Immunol. 125:2473-2477, 1980). Anti-IFNγ mAb is a rat antibody, produced by hybridoma R4-6A2, was obtained from American Type Culture Collection (ATCC) (Rockville, Md.) (Havell, J. Interfern Res. 6:489-497, 1986). Polyclonal rabbit anti-IFNγ serum was produced by immunization with recombinant murine IFNγ. Alkaline phosphatase conjugated goat anti-rabbit IgG was purchased by Southern Biotechnology (Birmingham, Ala.).

Synthetic peptides. L^{d} binding peptides pRL1a (IPGLPLSL, SEQ ID NO:1; Uenaka et al., J. Exp. Med. 180:1599-1607, 1994), p2Ca (LSPFPFDL, SEQ ID NO:2; Udaka et al., Cell 69:989-998, 1992) and T2H (ISTQNHRALDLVA, SEQ ID NO:3; Lurquin et al., Cell 58:293-303, 1989) were synthesized by standard solid-phase methods using F-moc chemistry (Atherton et al., J. Chem. Soc. Lond. Perkin. Trans. 1:538-543, 1981) in a peptide synthesizer (model 430A, Perkin-Elmer Applied Biosystems, Foster City, Calif.).

CTL clone. RL♂1 specific CTL clone B-24 was established from spleen cells of a BALB/c mouse bearing RL♂1 tumor. The CTL clone B-24 was maintained by weekly restimulation with mitomycin C (MMC)-treated RL♂1 stimulator cells and MMC-treated BALB/c splenic feeder cells in the presence of recombinant human IL-2 (Yokoi, T., 1997, Int. Immunol 9:1195-1201).

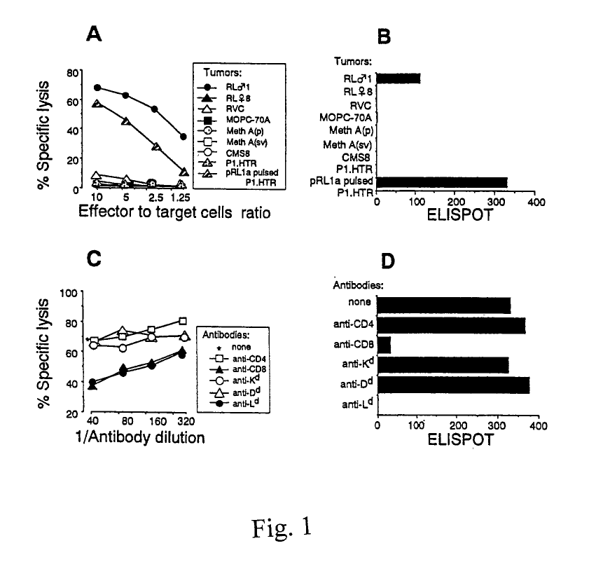

Cytotoxicity assay. Tumor cells were labeled by incubating 2×10⁶ cells with 2 MBq of Na₂⁵¹CrO₄, (New England Nuclear, Boston, Mass.) in 0.3 ml of medium for 90 min at 37° C. under 5% CO₂ in air. The cells were washed and used as targets. In direct assays, 10⁴ labeled target cells (100 μl) were incubated with increasing numbers of effector cells (100 μl) up to a maximum of 10⁵ effector cells (i.e., ratios of effector cells:target cells of 1.25:1 to 10:1; see FIG. 1A). In antibody blocking assays, serially diluted mAb (100 μl) was added to the mixture of effector cells and 10⁴ labeled target cells (100 μl). After incubation for 3.5 hr at 37° C. under 5% CO₂ in air, the supernatants were removed and their radioactivity was measured. The percentage of specific lysis was calculated by the following equation: (a-b)/(c-b)×100, where a is the radioactivity in the supernatant of target cells mixed with effector cells, b is the radioactivity in the supernatant of target cells incubated alone, and c is the radioactivity in the supernatant after lysis of target cells with 1% NP-40.

ELISPOT assay. The original ELISPOT assay for detecting cytokine producing cells (Czerkinsky et al., J. Immunol. Methods 110:29-36, 1988) was modified. Nitrocellulose disk membranes (14 mm diameter) and 96-well nitrocellulose membrane-based plates (Millipore S4510, Millipore Corp., Bedford, Mass.) were coated with anti-IFNγ mAb (R4-6A2) in 0.05 M bicarbonate buffer (pH 9.6) at 4° C. overnight. After washing, membranes were blocked with RPMI 1640 containing 10% FCS and 50 μM 2-mercaptoethanol for 60 min at 37° C. CTL were incubated with stimulator cells on the coated membrane in 96- or 24-well plates for 18 hr at 37° C. under 5% CO₂ in air. After culture, the membranes were thoroughly washed with distilled water and incubated with detection antibody for 90 min at 37° C. The IFNγ spots were developed by alkaline phosphatase-conjugated substrate kit (Bio-Rad, Hercules, Calif.) and counted with a dissecting microscope.

IFNγ ELISA. The culture supernatant of the stimulator cells incubated with CTL for 18 hr was assayed for IFNγ by sandwich ELISA. Anti-IFNγ mAb (R4-6A2) was used as the capture antibody and polyclonal rabbit anti-IFNγ antibody was used as the detection antibody. The readout was done by using horseradish peroxidase conjugated anti-rabbit IgG antibody (MBL, Nagoya, Japan) and O-phenylenediamine dihydrochloride as the substrate.

Construction of pMETRLakt and pMET7EGFP. The fragment of positions 116-1784 of RLakt (Wada et al., Cancer Res. 55:4780-4783, 1995) was amplified by RT-PCR using a forward primer 5′-TTGACTGCCCAGCTTGGGGGT-3′ (SEQ ID NO:4) and a reverse primer 5′-CATCCGAGAAACACATCAGGT-3′ (SEQ ID NO:5), digested with SalI and NotI, and ligated into pMET7 vector (provided by Dr. A. Shibuya, Tsukuba University, Tsukuba, Japan). The EGFP fragment was digested from pEGFP-N1 vector (Clontech, Palo Alto, Calif.) with SalI and NotI and then ligated into pMET7 vector.

Construction of cDNA library. mRNA was isolated from RL♂1 cells using QuickPrep mRNA Purification Kit (Amersham Pharmacia Biotech, Piscataway, N.J.) and first strand cDNA was synthesized using oligo (dT) primer. The cDNA were inserted into the Sal I and Not I sites of expression vector pMET7 (provided by Dr. A. Shibuya, Tsukuba University, Tsukuba, Japan) as described in the manufacturer's instructions of the SuperScript™ Plasmid System (GIBCO BRL, Rockville, Md.). The cDNA library was divided into pools of about 10,000 bacterial colonies.

Transfection and Screening. Recombinant plasmids were purified from bacteria using the Wizard Plus Series 9600™ DNA purification system (Promega Co., Madison, Wis.) and were transfected with plasmid pdl3027 containing the polyoma T antigen (Dailey and Basilico, J. Virol. 54:739-749, 1985) using lipofectamine into 1×10⁵ and 1×10⁴ CMS8 cells in 24- and 96-well plates respectively. After culture for 24 hr under 5% CO₂ in air, the cells were transferred onto anti-IFNγ mAb-coated nitrocellulose membranes in culture plates and then B-24 CTL were added. After culture overnight, the spots were detected.

DNA sequencing and homology search. DNA sequence analysis was performed using BigDye terminator cycle sequencing ready reaction kit (Applied Biosystems, Foster, Calif.) and a DNA sequencer ABI PRISM (Applied Biosystems). The computer search for sequence homology was performed by the publicly available BLAST software program on GenBank database (National Center for Biotechnology Information).

### Example 1

### Cytotoxicity and IFNγ ELISPOT by CTL Clone B-24

CTL clone B-24 was established from spleen cells of an RL♂1-bearing BALB/c mouse by repetitive stimulation with RL♂1 cells. Cytotoxicity and IFNγ ELISPOT were examined with CTL clone B-24. As shown in FIG. 1A and FIG. 1B, B-24 showed cytotoxicity and IFNγ ELISPOT against RL♂1 and pRL1 a peptide pulsed P1.HTR, but not BALB/c radiation-induced leukemia RL♀8, BALB/c RadLV-induced leukemia RVC, BALB/c mineral oil-induced myeloma MOPC-70A, BALB/c methylcholanthrene-induced fibrosarcomas Meth A and CMS8, or the peptide unpulsed P1.HTR. As shown in FIG. 1C and D, cytotoxicity and IFNγ ELISPOT by CTL clone B-24 was blocked by anti-CD8 mAb and anti-H-2L^{d} mAb, but not by anti-CD4 mAb, anti-H-2K^{d} mAb or anti-H-2D^{d} mAb. Thus B-24 was a CD8 CTL clone specific for pRL1a peptide epitope binding to H-2L^{d}.

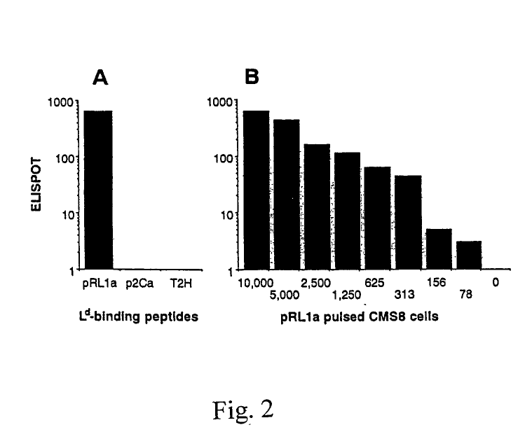

ELISPOTs produced by 5,000 B-24 cells in response to CMS8 stimulator cells pulsed with pRL1a and two other L^{d} binding peptides and to different numbers of pRL1a pulsed P1.HTR stimulator cells were examined in 96-well culture plates. As shown in FIG. 2A, 629 spots were observed against 10,000 CMS8 stimulator cells pulsed with pRL1a peptide, but no spot was observed against CMS8 cells pulsed with p2Ca or T2H. As shown in FIG. 2B, the number of spots were gradually decreased with the reduction of stimulator cells.

### Example 2

### Small and Large Scale ELISPOT Assays of CTL Clone B-24 in Response to CMS8 Cells Transfected with Recombinant RLakt Plasmid Mixed with Control EGFP Plasmid

The sensitivity and efficiency of large and small scale ELISPOT assays were investigated with the known RLakt as a model gene. RLakt codes for the peptide antigen pRL1a (Uenaka et al., J. Exp. Med. 180:1599-1607, 1994) recognized by CTL clone B-24.

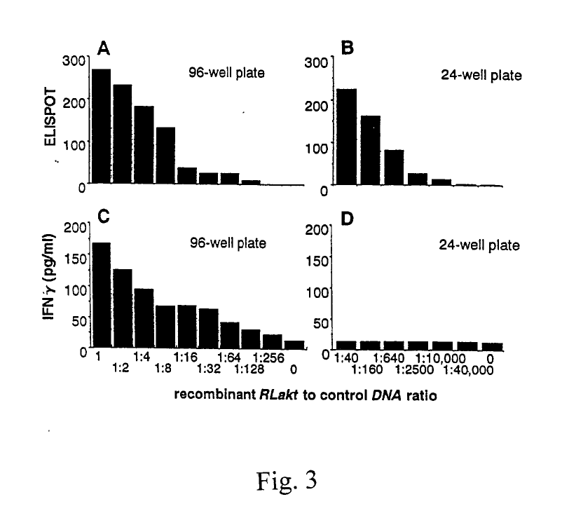

Conventional small scale ELISPOT assays were performed using purified recombinant RLakt plasmids mixed with control EGFP plasmids at various weight ratios were transfected into 1×10⁴ CMS8 cells at a total amount of 100 ng in 96-well culture plates. After 24 hours, the transfectants were collected and transferred onto anti-IFNγ mAb-coated nitrocellulose membrane-based 96-well culture plates. Next, 5,000 B-24 cells were added. After culture overnight, spots were detected. As shown in FIG. 3A, B-24 produced 268 spots against CMS8 cells transfected with 100 ng RLakt without EGFP plasmids. The number of spots were gradually decreased with decreasing amounts of RLakt plasmid. As few as 25 and 8 spots were observed with a lower ratios of RLakt: GFP of 1:64 and 1:128, respectively. No spots were observed with control EGFP alone.

Next, to augment sensitivity for detection of lower ratios of RLakt: EGFP (<1:100), large scale ELISPOT assays were performed. For these assays, a total amount of 3 μg of the mixture of the RLakt and EGFP plasmids was transfected into 1×10⁵ CMS8 cells in 24-well culture plates. After culture for 24 hr, the transfectants were collected and transferred onto anti-IFNγ mAb-coated nitrocellulose membranes of 14 mm diameter in 24-well culture plates. For detection of ELISPOT, 50,000 B-24 cells were added. After overnight culture, spots were counted. As shown in FIG. 3B, as many as 82, 27 and 14 spots were observed at RLakt: EGFP plasmid ratio of 1:640, 1:2,500 and 1:10,000, respectively. The number of spots with control EGFP plasmid alone were 4 in the depicted experiment and within a range between 0 to 5 in repeated experiments.

IFNγ ELISA with supernatant (100 μl) from culture of transfectants and B-24 cells in the small and large scale assays in 96- and 24-well plates similarly done without nitrocellulose membranes was performed using 96-well plates. A dose response curve similar to that of the ELISPOT assay was observed with supernatant from a 96-well plate (FIG. 3C), but no IFNγ was detected even in 1:40 ratio with supernatant from a 24-well plate (FIG. 3D).

### Example 3

### Identification of RLakt Gene by B-24 ELISPOT Screening of RL♂1 cDNA Library

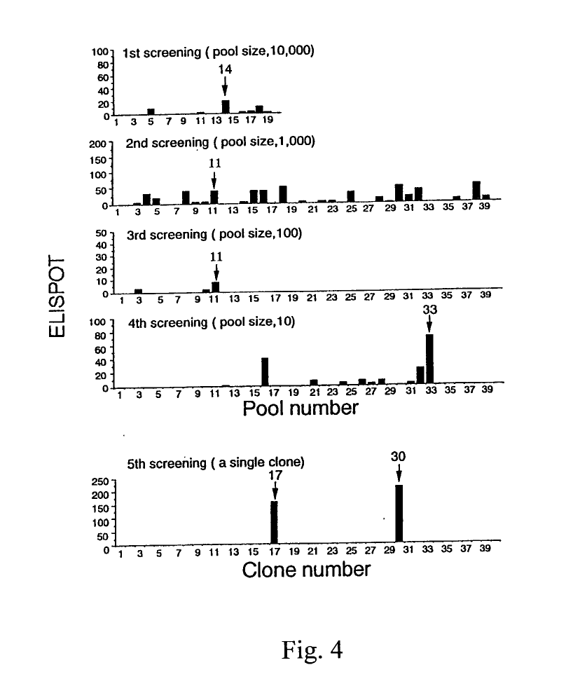

To evaluate sensitivity and efficiency of combined use of small and large scale ELISPOT assays for cDNA expression cloning, this method was applied for detection of the antigenic RLakt gene from a RL♂1 cDNA library. As shown in FIG. 4, ELISPOT screening was repeated five times in different sized pools of recombinant plasmids. The first two rounds of screening were done in large scale in 24-well culture plates and the last three rounds of screening were done in small scale in 96-well plates.

For the first round of screening, 10,000 colonies of bacterial colonies per pool were used. Twenty bacterial pools were prepared in total. Approximately 3 μg of plasmids purified from a bacterial pool were transfected into 1×10⁵ CMS8 cells. After incubation for 24 hr, the cells were collected by treating the plate with trypsin/EDTA and transferred onto anti-IFN γ mAb-coated nitrocellulose membranes in freshly prepared 24-well culture plates as described above. Then 50,000 B-24 cells were added to the wells. After overnight culture, the spots were detected. Three plasmid pools with more than 10 spots were obtained.

For the second round of screening, pool number 14 (20 spots) was selected and diluted to 1,000 bacterial colonies per pool. A total of 40 pools were prepared. Transfection and ELISPOT assays were conducted as described for the first round of screening. Thirteen pools with more than 10 spots were obtained.

For the third round of screening, pool number 11, which showed discernible large spots, was selected and diluted to 100 bacterial colonies per pool. A total of 40 pools were prepared. After purification of the plasmid DNA, 100-200 ng of plasmids per bacterial pool were transfected into 1×10⁴ CMS8 cells in 96-well plates. After incubation for 24 hr, the transfected cells were collected and transferred onto anti-IFNγ mAb-coated nitrocellulose membrane-based 96-well plates. Then 5,000 B-24 cells were added to each well. After overnight incubation, the spots were detected.

For the fourth round of screening, pool number 11 (8 spots) was selected and diluted to 10 bacterial colonies per pool. A total of 40 pools were prepared. Transfection and ELISPOT assays were conducted as described for the third round of screening. Three pools with more than 20 spots were obtained.

For the final round of screening, pool number 33 was chosen and diluted so that 40 wells were prepared with a single bacterial colony each. Two single plasmid clones were obtained, clone numbers 17 and 30.

### Example 4

### Determination of RLakt Insert in the Plasmid by RT-PCR

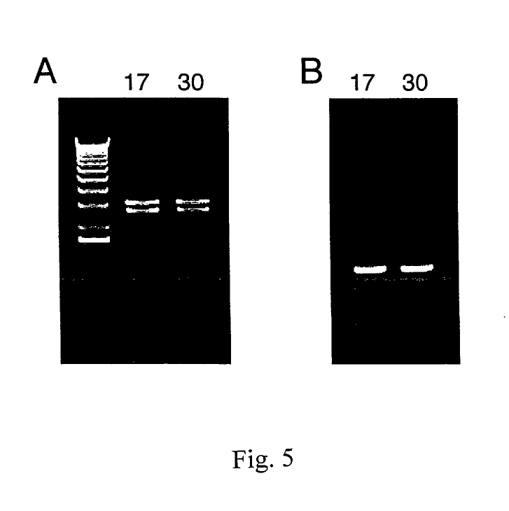

As shown in FIG. 5A, purified plasmid clone numbers 17 and 30 were subjected to Sal I and Not I digestion. The insert size was 2.6 kb, which is consistent with the size of full length RLakt mRNA as determined by northern blot analysis (Yokoi et al., Int. Immunol. 9:1195-1201, 1997). As shown in FIG. 5B, PCR of clones 17 and 30 using RLakt specific sense primer 5′-CAGCTTGGGGGTCTTTCAACAT-3′ (RLakt 125; SEQ ID NO:6) and anti-sense primer 5′-AGACACAATCTCCGCACCATAGA-3′ (RLakt 1020; SEQ ID NO:7) produced the predicted 896 bp band as analyzed by gel electrophoresis. A portion of the insert from plasmid clone number 30 was sequenced and confirmed as that of RLakt (Wada et al., Cancer Res. 55:4780-4783, 1995).

### Example 5

### ELISPOT Cloning of Meth A tumor Antigen Recognized by Cytotoxic T-lymphocytes from cDNA Expression Library

Materials and Methods

Tumors and cell lines. Meth A, CMS4, CMS5a, CMS5j, CMS8, CMS9 and CMS13 are methylcholanthrene-induced sarcomas in BALB/c mice (Srivastava, et al., Proc. Natl. Acad. Sci. 83(10):3407-11. 1986;, DeLeo et al., J. Exp. Med. 146:720-734, 1977; Palladino et al., Cancer Res. 47:5704-5709, 1987). Parental Meth A, Meth A (p) is resistant to CTL lysis. Meth A (sv) is a CTL lysis-sensitive variant line that occurred during in vivo passages of Meth A (p) and provided by Dr. H. Shiku (Mie University School of Medicine, Mie, Japan). RL♂1 is a radiation-induced leukemia in BALB/c mouse (Nakayama et al., Cancer Res. 44:5138-5144, 1984). P815 is a methylcholanthrene-induced mastocytoma in a DBA/2 mouse (Dunn and Potter, J. Natl. Cancer Inst. 18:587-601, 1957). T1.1.1 and T4.8.3 are derivatives of L cells (H-2^{k}) transfected with the H-2L^{d} and H-2D^{d} gene, respectively (Margulies, et al., J. Immunol. 130(1):463-70, 1983).

Antibodies. Anti-L3T4 (CD4) mAb, a rat antibody of the IgG2b immunoglobulin class, produced by hybridoma GK1.5 (Dialynas et al., J. Immunol. 131:2445-2451, 1983), was provided by Dr. F. Fitch (University of Chicago, Chicago, Ill.), anti-Lyt-2.2 (CD8) mAb (Nakayama et al., Cancer Res. 44:5138-5144, 1984), a mouse antibody of the IgG2ₐ class, produced by hybridoma 19/178 was provided by Dr. G. Hammerling (Memorial Sloan-Kettering Cancer Center, New York). Anti-H-2K^{d} and anti-H-2D^{d} are mouse antibodies produced by hybridomas HB159 and HB102, respectively. Anti-H-2L^{d} mAb is a mouse IgG2ₐ antibody produced by hybridoma 30-5-7 (Ozato et al., J. Immunol. 125:2473-2477, 1980). Anti-IFNγ mAb, a rat antibody of the IgG1 class, produced by hybridoma R4-6A2 was obtained from American Type Culture Collection (ATCC) (Rockville, Md.) (Havell, J. Interfern Res. 6:489-497, 1986). Polyclonal rabbit anti-IFNγ serum was produced by immunization with recombinant murine IFNγ (Uenaka ELISPOT). Alkaline phosphatase conjugated goat anti-rabbit IgG was purchased from Southern Biotechnology (Birmingham, Ala.).

Peptides synthesis. Peptides were synthesized by standard solid-phase methods using F-moc chemistry in a peptide synthesizer (model AMS422; MS Techno Systems Co., Osaka, Japan). Cleavage of the peptide from the resin and removal of side chain protecting groups were carried out by 95% trifluoroacetic acid (TFA).

Deletion constructions. Deleted S35 cDNA were amplified by PCR using pCAGGS-S35 plasmid for template. PCR amplification was performed at 94° C. for 1 min followed by 30 cycles of 94° C. for 1 min, 55° C. for 90s, and 72° C. for 1 min. The PCR fragments were cloned into the expression vector pCI-neo with EcoRI and Sal I sites (Promega Co. Madison, Wis.).

Site-directed Mutagenesis. Site-directed mutagenesis, was done using non-PCR quickchange kits (Stratagene, Ceder Creek, Tex.). Primers S35-S683SITEMU [5′-CTCTACGCTTTCTTCTCGATCACCATT-3′ (SEQ ID NO:8)] and S35AS717-SITEMU [5′-CTGGGAGTAATGGTGATCGAGAAGAAA-3′ SEQ ID NO:9)] were used to generate the mutations (A to C) at nucleotide 699 and (G to A) at nucleotide 701. The mutant was sequenced to confirm the orientation and nucleotide sequence.

cDNA library construction. mRNA was isolated from Meth A (sv) cells using QuickPrep mRNA Purification Kit (Amersham Pharmacia Biotech, Piscataway, N.J.) and first strand cDNA was synthesized using random hexamer primer (GIBCO BRL, Rockville, Md.). The cDNA were inserted into the EcoRI sites of expression vector pCAGGS (provided by Dr. A. Shibuya, University of Tsukuba, Tsukuba, Japan) according to the manufacture's (SuperScript™ Plasmid Choice System, GIBCO BRL). The recombinant plasmids were electroporated into DH10B Escherichia coli bacteria (GIBCO BRL). The cDNA library was divided and stocked as pools containing about 10,000 bacterial colonies.

Reverse Transcription-PCR. mRNA was purified from cells using the QuickPrep Micro mRNA purification Kit (Amersham Pharmacia). mRNA was reverse-transcribed into single-strand cDNA using Molony murine leukemia reverse transcriptase and oligo(dT)₁₅ as a primer (Amesham Pharmacia), and cDNAs were tested for integrity by amplification of β-actin transcripts in a 30 cycle reaction. RT-PCR was performed by 35 amplification cycles and the products were analyzed by agarose gel electrophoresis.

Transfection. Recombinant plasmids were purified by Wizard Plus Series 9600™ DNA purification system (Promega, Co., Madison, Wis.) after amplification of bacteria and transfected into 1×10⁵ or 1×10⁴ cells 24- or 96-well plates, respectively, with plasmid pdl3027 containing the polyoma T antigen (Dailey and Basilico, J. Virol. 54:739-749, 1985) using lipofectamine (GIBCO BRL, Rockville, Md.). After culture for 24 hr, the transfected cells were collected and plated onto anti-IFNγ mAb-coated nitrocellulose membranes in culture plates. AT-1 CTL were then added for ELISPOT assay.

DNA sequencing and homology search. DNA sequence was determined using BigDye terminator cycle sequencing ready reaction kit (Perkin-Elmer Applied Biosystems, Foster City, Calif.) and a DNA sequencer ABI PRISM (Perkin-Elmer Applied Biosystems, Foster City, Calif.). The computer search for sequence homology was performed by the program BLAST on GenBank database.

IFNγ ELISA. CTL were stimulated with targets for 18 hr. and IFNγ in the culture supernatant was assayed by the sandwich ELISA. Anti-IFNγ mAb (R4-6A2 was used) as the capture antibody and polyclonal rabbit anti-IFNγ antibody as the detection antibody. Horseradish peroxidase conjugated anti-rabbit IgG antibody (MBL, Nagoya, Japan) and O-phenylenediamine dihydrochloride were used for indicator.

CTL clone. Spleen cells (5×10⁴) from a Meth A (sv)-immunized (BALB/cXC57BL/6)F₁ (CB6F₁) mouse were cultured with 5×10⁶ cells of mitomycin C (MMC)-treated Meth A (sv) in 25-cm flasks (353014, Becton Dickinson, Franklin Lakes, N.J.) at 37° C. in a 5% CO₂ atmosphere. The culture medium was RPMI 1640 supplemented with 10% heat-inactivated FCS, 2 mM glutamine, 100U of penicillin, 100 μg/ml of streptomycin and 50 μM 2-mercaptoethanol. The cells were restimulated with MMC-treated Meth A (sv) in the presence of 20 U human recombinant IL-2 (rIL-2, Takeda Pharmaceutical Industries, Osaka, Japan). After the second in vitro stimulation, Meth A specific 13 CTL clones were established by limiting dilution. Recognition of 11 clones was D^{d} restricted and that of 2 clones was K^{d} restricted. One of D^{d} restricted CTL clone designated AT-1 was used.

Cytotoxicity assay. Tumor cells were labeled by incubating 2×10⁶ cells with 2 MBq of Na₂⁵¹CrO₄ (New England Nuclear, Boston, Mass.) in 0.3 ml of medium for 90 min at 37° C. under a 5% CO₂ in air. The cells were washed and used as targets. For peptide sensitization, labeled target cells were incubated with peptides at 37° C. for 30 min at room temperature. In direct assays, 10⁴ labeled target cells (100 μl) were incubated with effector cells (100 μl) cells. In antibody blocking assays, serially diluted mAb (100 μl) was added to the mixture of effector cells and 10⁴ labeled target cells (100 μl). After incubation for 4 hr at 37° C. under a 5% CO₂ in air, the supernatants were removed and their radioactivity was measured. The percentage of specific lysis was calculated by the following equation: (a−b)×100/(c−b), where a is the radioactivity in the supernatant of target cells mixed with effector cells, b is that in the supernatant of target cells incubated alone, and c is that in the supernatant after lysis of target cells with 1% NP-40.

ELISPOT assay. Nitrocellulose disk membranes (14 mm diameter) and 96-well nitrocellulose membrane-based plates (Millipore S4510, Millipore Corp., Bedford, Mass.) were coated with anti-IFNγ mAb (R4-6A2) in 0.05 M bicarbonate buffer (pH 9.6) at 4° C. overnight. After washing, membranes were blocked with RPMI 1640 containing 10% FCS and 50 μM 2-mercaptoethanol for 60 min at 37° C. CTL were incubated with stimulator cells on the coated membrane in 24- or -96well plates for 18 hr at 37° C. under a 5% CO₂ in air. After culture, the membranes were thoroughly washed with distilled water and incubated with polyclonal rabbit anti-IFNγ antibody for 90 min at 37° C. The IFNγ spots were developed by alkaline phosphatase-conjugated anti-rabbit IgG antibody and substrate kit (Bio-Rad, Hercules, Calif.) and counted with a dissecting microscope.

Results

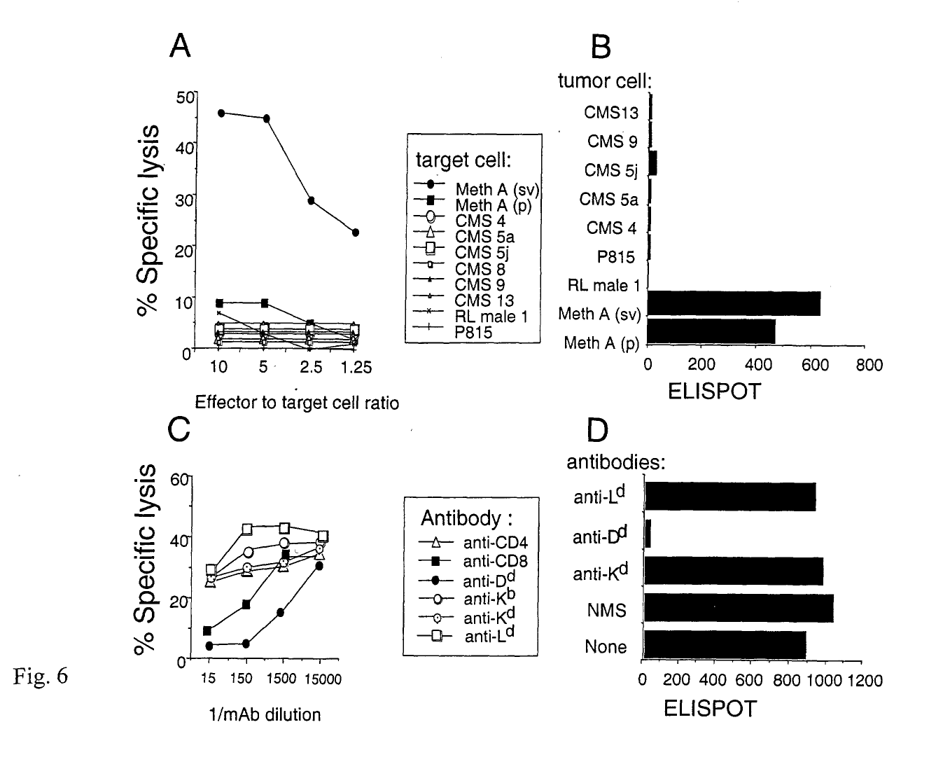

Cytotoxicity and IFNγ ELISPOT by CTL clone AT-1. CTL clone AT-1 was established from Meth A (sv)-immunized CB6F, spleen cells by repetitive in vitro stimulation with MMC-treated Meth A (sv) cells. As shown in FIG. 6B, AT-1 specifically recognized parental Meth A, Meth A (p) and Meth A variant sensitive for CTL lysis, Meth A (sv) targets by IFNγ ELISPOT assay. Meth A (sv) but not Meth A (p) was lysed in standard 4 hr ⁵¹Cr release assay (FIG. 6A). Cytotoxicity in FIGS. 6A and C was determined by standard 4 hr ⁵¹Cr-release assay. The number of target cells was 1×10⁴. Effector to target cell ratio in FIG. 6C was 10. IFNγ ELISPOT was determined after 24 hr culture. The number of AT-1 cells in FIGS. 6B and D was 1×10⁴. The number of target cells was 1×10⁴. The antibody dilution in FIG. 6D was 1/100. Failure of Meth A (p) lysis by CTL appeared to be due to intrinsic resistance to lysis because no lysis of those cells was also observed even by anti-allogenic H-2^{d} CTL (Levey, D. L., et al., Cancer Immunity 1:5, 2001. None of BALB/c methylcholanthrene sarcomas CMS4, CMS5a, CMS5j, CMS8, CMS9 and CMS13, BALB/c radiation leukemia RL male 1, or DBA/2 mastocytoma P815 was recognized. As shown in FIG. 6C and D, AT-1 cytotoxicity and IFNγ ELISPOTs for Meth A (sv) were blocked by anti-CD8 mAb and anti-H-2D^{d} mAb, but not anti-CD4 mAb, anti-H-2K^{d} mAb or anti-H-2L^{d} mAb.

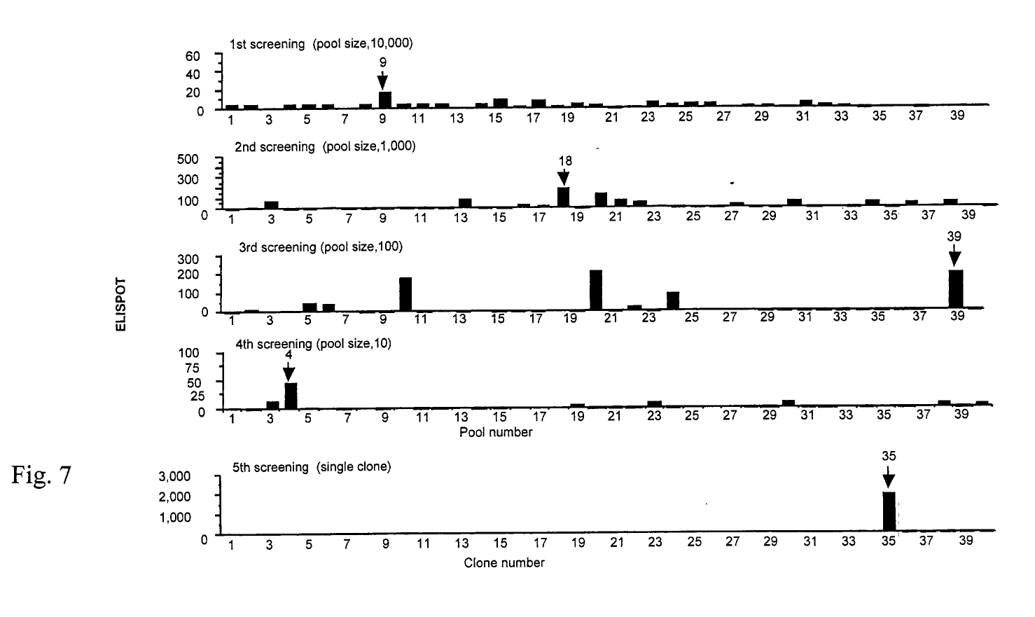

Cloning of Meth A antigen recognized by AT-I from Meth A (sv) cDNA library by ELISPOT assays. For cloning/the gene that coded for the antigen recognized by AT-1, cDNA library from Meth A (sv) mRNA was prepared. 4×10⁵ cDNA clones were screened by ELISPOT assay according to the method previously described. In the first round of screening, 40 cDNA pools each containing 10,000 bacterial colonies were prepared. 3 μg DNA from each pool and polyoma T were cotransfected into 1×10⁵ CMS5a cells. After incubation for 24 hr, the transfectants were collected, transferred onto anti-IFNγ mAb coated nitrocellulose membranes in newly prepared 24-well culture plates and 50,000 AT-1 CTL were added. As shown in FIGS. 7A and B, 18 spots were observed on the pool number 9, after overnight culture. For the second round of screening, bacteria in the pool number 9 were diluted into 40 pools each containing 1,000 bacterial colonies transfected and assayed as above. For the third round of screening, bacteria in the positive pool number 18 were diluted into 40 pools each containig 100 bacterial colonies, transfected and assayed. For the fourth and fifth round of screenings, 100 ng DNA and polyoma T were cotransfected into 1×10⁴ CMS5a cells. After the incubation, the cells were transferred onto an anti-IFNγ mAb coated nitrocellulose membrane-based 96-well plate and AT-1 by ELISPOTs were detected. Finally, cDNA clone S35 (SEQ ID NO: 13) was obtained. AT-1 was stimulated with CMS5a, CMS8 and CMS13, or H-2D^{d}- but not H-2L^{d}-transfected L cells transfected with S35.

Identification of the epitope peptide recognized by AT-1 in S35. cDNA clone S35 (SEQ ID NO:13) was 937 bp long. The open reading frame of S35 that had a peptide recognized by the AT-1 T cell clone is the third reading frame. To identify the epitope peptide recognized by AT-1 CTL, truncated S35 was prepared and transfected into CMS8 cells. IFNγ production from AT-1 by stimulation with transfected CMS8 cells was assayed after culture for 24 hr by ELISA.

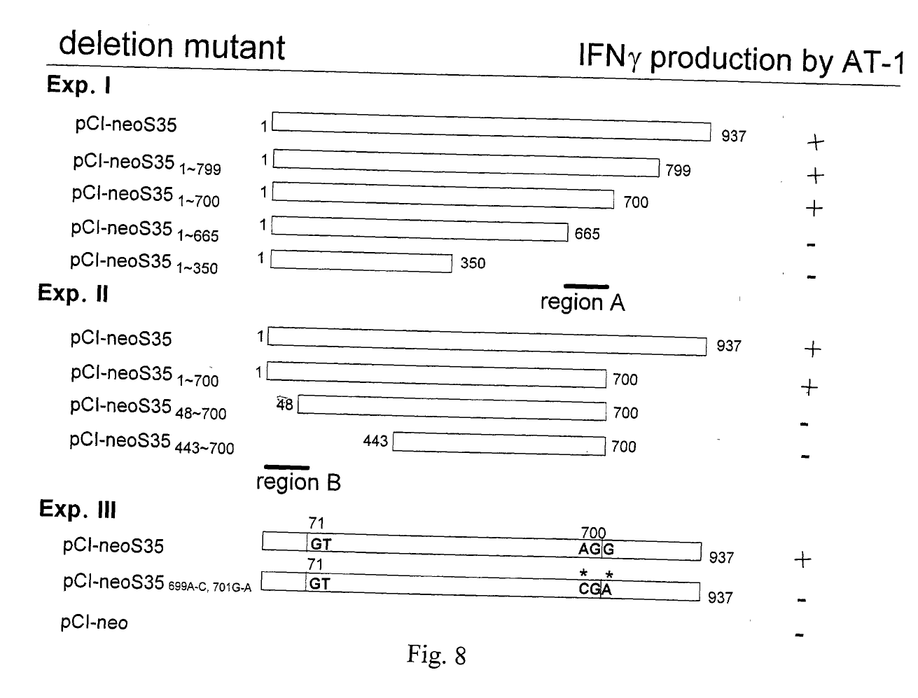

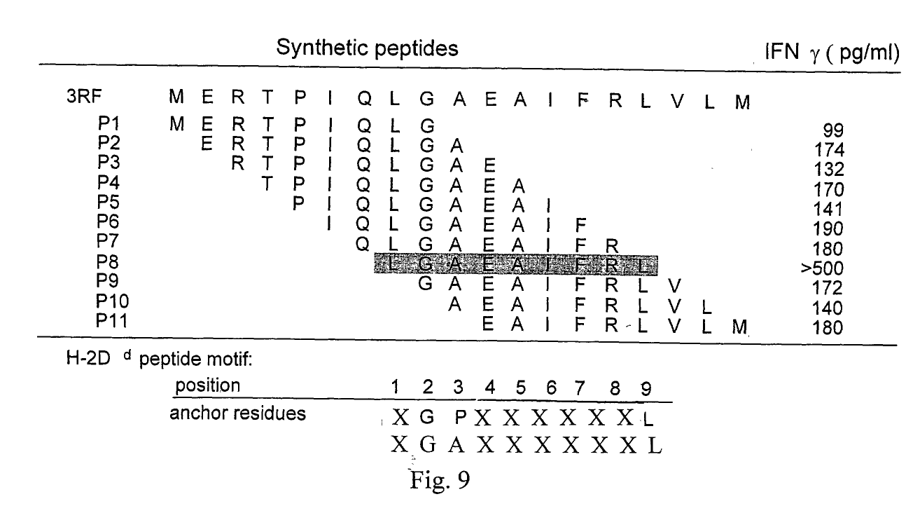

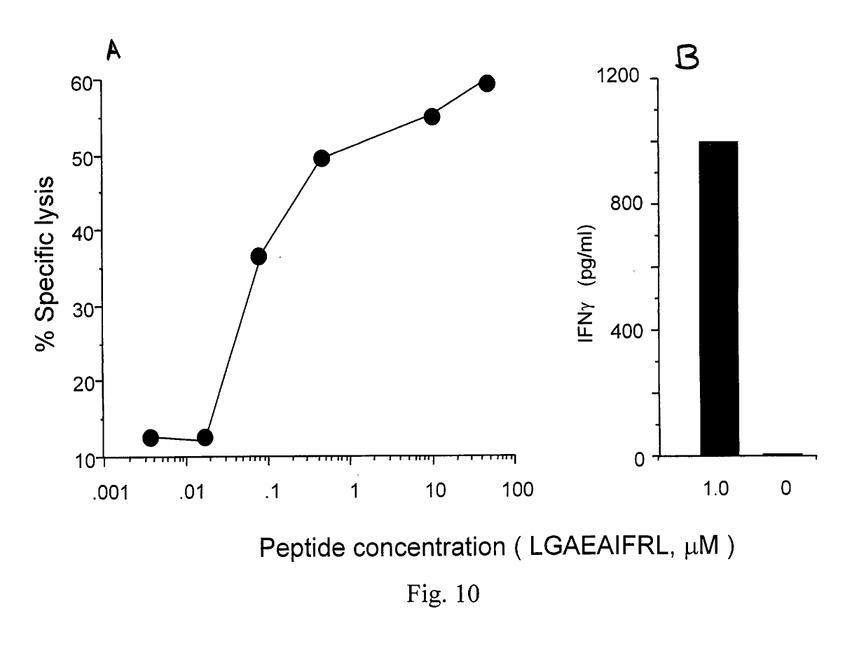

As shown in FIG. 8 Exp. I, 3′ truncated S35, nt 1-799, nt 1-700 but not nt 1-665 stimulated AT-1 for IFNγ production. Therefore, we synthesized 9 mer overlapping peptides covering the region A spanning nt 665-700 and tested to stimulate AT-1 for IFNγ production by the peptide pulsed CMS8 cells. However, no stimulation was observed with those synthetic peptides. We then prepared 5′ truncated S35, nt 48-700 and nt 443-700 and tested for their stimulatory activity (FIG. 8, Exp. II). No stimulation was observed with either mutants. We synthesized 9 mer overlapping peptides covering the region B spanning nt 1-48 and tested to stimulate AT-1 for IFNγ production by the peptide pulsed CMS8 as above. As shown in FIG. 9, stimulation was observed with the peptide LGAEAIFRL (SEQ ID NO:10). FIG. 9 also shows H-2D^{d} peptide motifs XGPXXXXXL (SEQ ID NO: 26) and XGAXXXXXL (SEQ ID NO:27). The sequence MERTPIQLGAEAIFRLVLMW (SEQ ID NO:14) is a peptide fragment that includes the end of the open reading frame. AT-1 CTL lysis for the peptide pulsed CMS8 was detected at a 20-30 nM peptide concentration (FIG. 10). Meth A (sv) lysis by AT-1 CTL was completely inhibited by the peptide pulsed P815.

Above results indicated that the downstream sequence spanning nt 665-700 was necessary for the expression of the epitope peptide LGAEAIFRL (SEQ ID NO: 10) corresponding to nt 32-58. There were typical splicing donor and acceptor sites at nt 71 and 72, and nt 699 and 700, respectively, in S35. To investigate the requirement of splicing between those sites for the epitope expression, we tested AT-1 stimulation by CMS8 transfected with S35 mutants that was introduced with A to C for nt 699 and G to A for nt 700 to disrupt the splicing acceptor site. No stimulation was observed (FIG. 8 Exp. III).

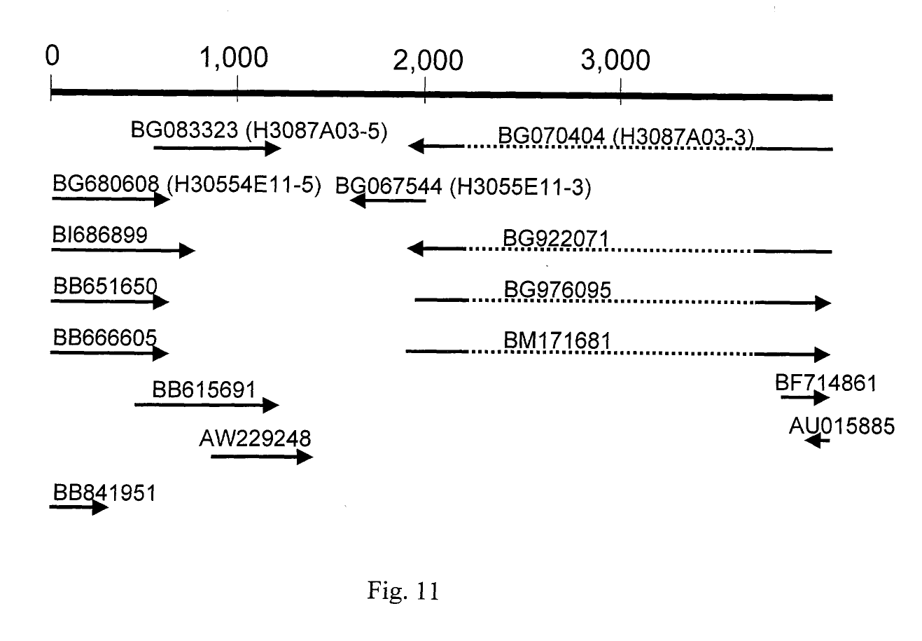

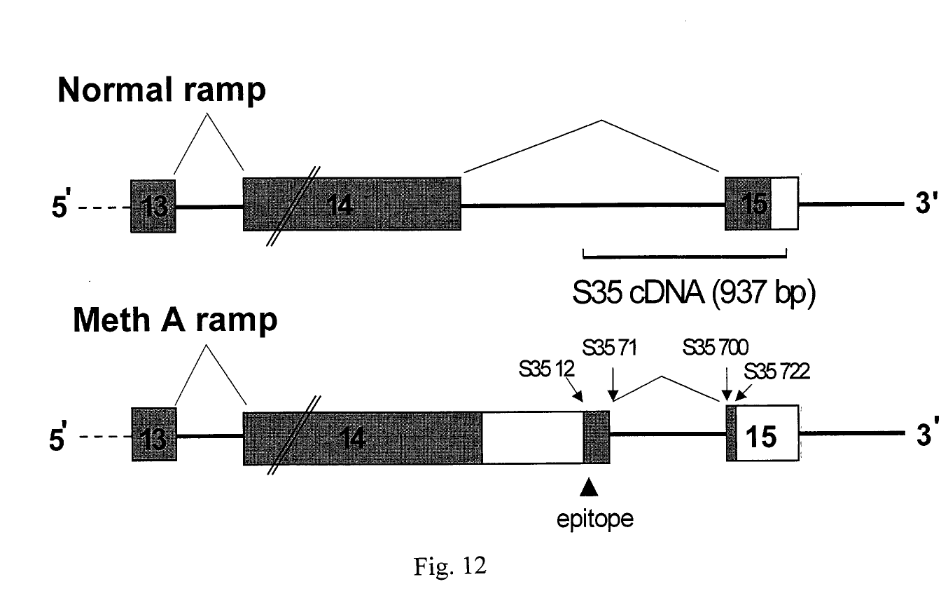

Analysis of cDNA that coded for the antigen recognized by AT-1 CTL. According to a GenBank BLAST search, nt 700-811 of S35 (937 bp long ) showed 86% homology to a region of human retinoic acid-regulated nuclear matrix-associated protein (ramp) gene, suggesting that S35 is a part of mouse homologue of ramp (Cheung et al., J. Biol. Chem. 276, 17083-91, 2001). The region of nt 1-699 of S35 showed no homology with any sequence. To obtain full-length cDNA, RT-PCR was carried out using human ramp nt 124-145 as 5′-primer and S35 nt 923-937 as 3′-primer with Meth A (p) cDNA. The ˜2.7 Kb PCR product was obtained and cloned. The nucleotide sequence of cloned products and the deduced amino acid showed homology of 85% and 89%, respectively, to human ramp. Subsequently, an even longer clone with 3805 bp, spanning exon 1 to 15 corresponding to those in human ramp was obtained. A number of mouse ESTs with homology for this sequence were identified. In ESTs, selective deletion spanning nt 2207 to 3568 was observed, suggesting that this region was intronic sequence (FIG. 11). Sequence homology indicated that this intron was between exon 14 and 15. The splicing donor site GT at nt 71 and nt 72 corresponded to nt 2939 and nt 2940. The splicing acceptor site AG at nt 699 and nt 700 corresponded to nt 3567 and nt 3568. RT-PCR analysis showed that most of the ramp mRNA underwent normal splicing between exon 14 and 15 in either Meth A (p) or Meth A (sv). However, RT-PCR product that lacked S35 nt 71-700 was also obtained. These results indicated that there is expressed ramp mRNA in Meth A in which exon 14 is expressed as shown in FIG. 12. A newly created open reading frame starting from S35 nt12 to S35 nt 722 harbored the epitope sequence.

### Example 6

### Determination of the Human Homolog of the Mouse Ramp Protein

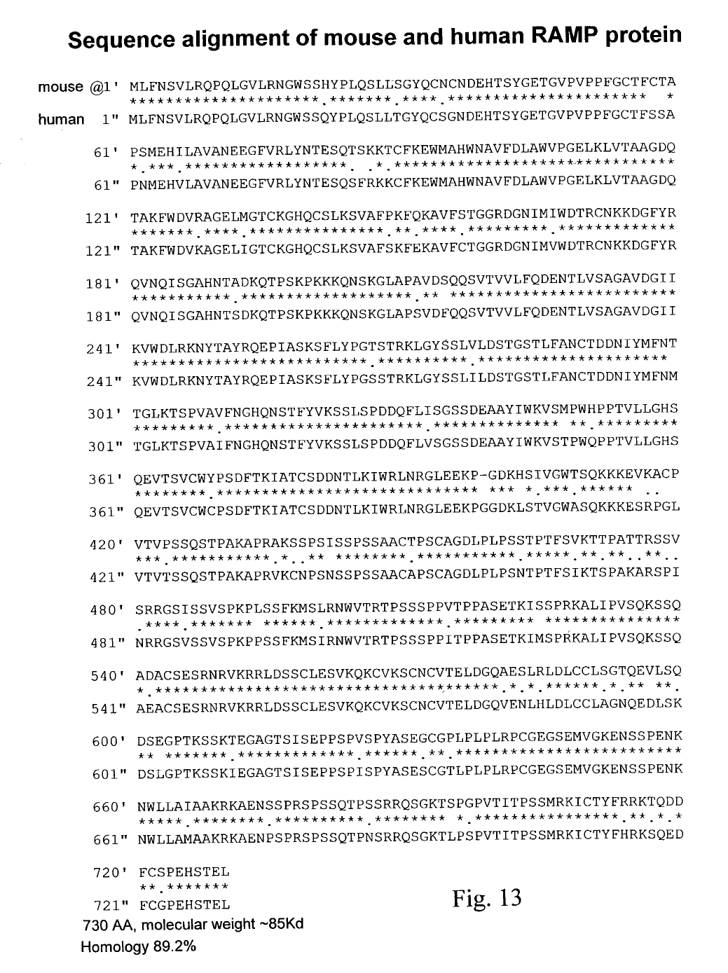

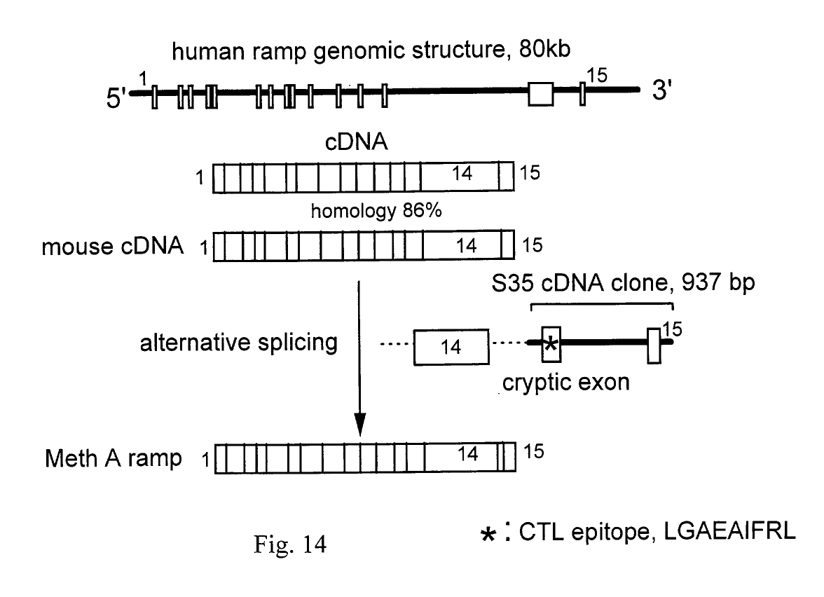

The human homolog (SEQ ID NO: 12) of the mouse ramp protein (SEQ ID NO:11) was identified and both are shown in FIG. 13. The human ramp sequence corresponds to Genbank No: NM_{—}016448. The human protein was found to have 730 amino acids and a molecular weight of approximately 85 Kd and was determined to have an 89.2% homology with the murine antigenic protein. The cDNA sequence of the human ramp protein was found to have 86% homology to the cDNA sequence of the murine ramp gene (FIG. 14).

### Example 7

### Investigation of Human Ramp as a Cancer-Associated Antigen

Expression of Human Ramp Antigen in Normal Tissues and Tumors

mRNA expression for the ramp gene is examined by RT-PCR, using a panel of normal mouse tissues and tumors. mRNA expression in normal tissues is examined and the expression pattern of the ramp gene in tumor tissues is also examined. Tissues in which mRNA expression is examined include, but are not limited to: brain, fetal brain, salivary gland, esophagus, thymus, heart, lung liver, fetal liver, spleen, lymph node, adrenal gland, kidney pancreas, stomach, duodenum, small intestine, colon, bladder, uterus, ovary, testis, placenta, bone marrow, and breast.

### Example 8

### Preparation of Recombinant Human Ramp Cancer-Associated Antigens

To facilitate screening of mice or patients' samples for cancer associated antigens, recombinant ramp proteins are prepared according to standard procedures. In one method, the clones encoding ramp antigen are subcloned into a baculovirus expression vector, and the recombinant expression vectors are introduced into appropriate insect cells. Baculovirus/insect cloning systems are preferred because post-translational modifications are carried out in the insect cells. Another preferred eukaryotic system is the Drosophila Expression System from Invitrogen. Clones which express high amounts of the recombinant protein are selected and used to produce the recombinant proteins.

Alternatively, the cancer associated ramp antigen clones are inserted into a prokaryotic expression vector for production of recombinant proteins in bacteria. Other systems, including yeast expression systems and mammalian cell culture systems also can be used.

### Example 9

### Preparation of Antibodies to Human Ramp Cancer-Associated Antigens

The recombinant cancer associated ramp antigens produced as in Example 8 above are used to generate polyclonal antisera and monoclonal antibodies according to standard procedures. The antisera and antibodies so produced are tested for correct recognition of the cancer associated ramp antigens. These antibodies can be used for experimental purposes (e.g. localization of the cancer associated antigens, immunoprecipitations, Western blots, etc.) as well as diagnostic purposes (e.g., testing extracts of tissue biopsies, testing for the presence of cancer associated antigens).

### Example 10

### Expression of Human Ramp Cancer-Associated Antigens in Cancers of Similar and Different Origin

The expression of ramp is tested in a range of tumor samples to determine which other malignancies can be diagnosed and/or treated by the methods described herein. Tumor cell lines and tumor samples are tested for ramp expression, preferably by RT-PCR according to standard procedures. Northern blots also are used to test the expression of ramp. Antibody based assays, such as ELISA and western blot, also can be used to determine protein expression.

The results generated from the foregoing experiments confirm the use of ramp nucleic acids and/or polypeptides in diagnostic (e.g. determining the existence of cancer, determining the prognosis of a patient undergoing therapy, etc.) and therapeutic methods (e.g., vaccination, etc.).

### Example 11

### Identification of the Portion of Human Ramp Cancer-Associated Polypeptide Encoding an Antigen

To determine if the human ramp described above can provoke a cytolytic T lymphocyte response, the methods of the invention are followed as described herein and/or the following method is performed. CTL clones are generated by stimulating the peripheral blood lymphocytes (PBLs) of a patient (or mouse) with autologous normal cells transfected with a clone encoding human ramp polypeptide or with irradiated PBLs loaded with synthetic peptides corresponding to the putative protein and matching the consensus for the HLA class I molecules to localize an antigenic peptide within the cancer associated antigen clone (see, e.g., Knuth et al., Proc. Natl. Acad. Sci. USA 81:3511-3515, 1984; van der Bruggen et al., Eur. J. Immunol.24:3038-3043, 1994). These CTL clones are screened for specificity against COS cells transfected with the ramp clone and autologous HLA alleles as described by Brichard et al. (Eur. J. Immunol. 26:224-230, 1996). CTL recognition of ramp is determined by ELISPOT as described above, by measuring release of TNF from the cytolytic T lymphocyte or by ⁵¹Cr release assay (Herin et al., Int. J. Cancer 39:390-396, 1987). If a CTL clone specifically recognizes a transfected COS cell, then shorter fragments of the ramp clone transfected in that COS cell are tested to identify the region of the gene that encodes the peptide. Fragments of the ramp clone are prepared by PCR or other standard molecular biology methods. Synthetic peptides are prepared to confirm the exact sequence of the antigen.

Optionally, shorter fragments of ramp cDNA are generated by PCR. Shorter fragments are used to provoke TNF release or ⁵¹Cr release as above.

Synthetic peptides corresponding to portions of the shortest fragment of the ramp clone which provokes TNF release are prepared. Progressively shorter peptides are synthesized to determine the optimal ramp tumor rejection antigen peptides for a given HLA molecule.

A similar method is performed to determine if the ramp contains one or more HLA class II peptides recognized by T cells. One can search the sequence of the ramp polypeptide for HLA class II motifs as described above. In contrast to class I peptides, class II peptides are presented by a limited number of cell types. Thus for these experiments, dendritic cells or B cell clones which express HLA class II molecules preferably are used.

Equivalents

Those skilled in the art will recognize, or be able to ascertain using no more than routine experimentation, many equivalents to the specific embodiments of the invention described herein. Such equivalents are intended to be encompassed by the following claims.

All references disclosed herein are incorporated by reference in their entirety.

We claim:

1. A method for identifying a nucleic acid molecule encoding an epitope that specifically binds to a T cell receptor on a T cell when presented by a HLA molecule, comprising
providing a T cell having a T cell receptor that binds the epitope,
providing a population of antigen presenting cells containing a library of nucleic acid molecules, wherein the cells express HLA molecules that present the epitope,
coculturing the antigen presenting cells with the T cell for a time sufficient for the T cell receptor to bind an epitope encoded-by the library of nucleic acid molecules,
detecting a factor secreted by the T cell in response to the T cell receptor binding using an ELISPOT assay, and
correlating the secretion of the factor with the presence of the nucleic acid molecule encoding the epitope in the library.

2. The method of claim 1, wherein the library of nucleic acid molecules is provided in an expression vector.

3. The method of claim 1, wherein the step of detecting the presence of the secreted factor comprises capturing the secreted factor on a solid support.

4. The method of claim 1, wherein the antigen presenting cells are cocultured with the T cell for less than 36 hours.

5. The method of claim 4, wherein the antigen presenting cells are cocultured with the T cell for less than 24 hours.

6. The method of claim 5, wherein the antigen presenting cells are cocultured with the T cell for less than 18 hours.

7. The method of claim 1, wherein the secreted factor is interferon-γ.

8. The method of claim 1, wherein the secreted factor is tumor necrosis factor-α.

9. The method of claim 1, wherein the secreted factor is detected by binding to an immobilized capture antibody.

10. The method of claim 9, wherein the capture antibody is immobilized on a membrane.

11. The method of claim 10, wherein the membrane is a nitrocellulose membrane.

12. The method of claim 1, wherein the secreted factor is detected by binding of a detection antibody.

13. The method of claim 12, wherein the detection antibody is conjugated to an enzyme.

14. The method of claim 13, wherein the enzyme is selected from the group consisting of alkaline phosphatase, horseradish peroxidase and glucose oxidase.

15. A method for identifying an antigen that specifically binds to a T cell receptor on a T cell, comprising
providing a T cell having a T cell receptor that binds the antigen,
providing a population of candidate antigens,
coculturing the candidate antigens with the T cell for a time sufficient for the T cell receptor to bind an antigen,
detecting a factor secreted by the T cell in response to the T cell receptor binding using an ELISPOT assay, and
correlating the secretion of the factor with the presence of the antigen.

16. The method of claim 15, wherein the candidate antigens are presented by antigen presenting cells.

17. The method of claim 15, wherein the candidate antigens are peptides.

18. The method of claim 17, wherein the peptides are a library of random or semi-random peptides.

19. The method of claim 17, wherein the peptides are peptides derived from an antigenic protein.

20. The method of claim 16, wherein the antigen presenting cells are tumor cells.

21. The method of claim 16, wherein the antigen presenting cells are cells infected with a microorganism.

22. The method of claim 15, wherein the candidate antigens are presented as tetrameric complexes of HLA molecules and antigens.

23. The method of claim 15, wherein the step of detecting the presence of the secreted factor comprises capturing the secreted factor on a solid support.

24. The method of claim 15, wherein the antigen presenting cells are cocultured with the T cell for less than 36 hours.

25. The method of claim 24, wherein the antigen presenting cells are cocultured with the T cell for less than 24 hours.

26. The method of claim 25, wherein the antigen presenting cells are cocultured with the T cell for less than 18 hours.

27. The method of claim 15, wherein the secreted factor is interferon-γ.

28. The method of claim 15, wherein the secreted factor is tumor necrosis factor-α.

29. The method of claim 15, wherein the secreted factor is detected by binding to an immobilized capture antibody.

30. The method of claim 29, wherein the capture antibody is immobilized on a membrane.

31. The method of claim 30, wherein the membrane is a nitrocellulose membrane.

32. The method of claim 15, wherein the secreted factor is detected by binding of a detection antibody.

33. The method of claim 32, wherein the detection antibody is conjugated to an enzyme.

34. The method of claim 33, wherein the enzyme is selected from the group consisting of alkaline phosphatase, horseradish peroxidase and glucose oxidase.

35. A kit comprising
a solid support for capturing factors secreted by T cells in response to antigen binding,
a container containing a capture antibody that binds the secreted factors,
a container containing a detection antibody that binds the secreted factors once bound to the capture antibody, and
instructions for using the solid support, capture antibody and detection antibody for the idenfication of an antigen that is specifically bound by a T cell.

36. The kit of claim 35, wherein the detection antibody is detectably labeled.

37. The kit of claim 35, wherein the detection antibody is labeled with an enzyme.

38. The kit of claim 37, further comprising a detectable enzyme substrate.


SEQUENCE LISTING (28 sequences)

SEQ ID NO: 1 - protein, 8 aa, Mus musculus
IPGLPLSL

SEQ ID NO: 2 - protein, 8 aa, Mus musculus
LSPFPFDL

SEQ ID NO: 3 - protein, 13 aa, Mus musculus
ISTQNHRALDLVA

SEQ ID NO: 4 - DNA, 21 bp, Mus musculus
ttgactgcccagcttgggggt

SEQ ID NO: 5 - DNA, 21 bp, Mus musculus
catccgagaaacacatcaggt

SEQ ID NO: 6 - DNA, 22 bp, Mus musculus
cagcttgggggtctttcaacat

SEQ ID NO: 7 - DNA, 23 bp, Mus musculus
agacacaatctccgcaccataga

SEQ ID NO: 8 - DNA, 27 bp, Mus musculus
ctctacgctttcttctcgatcaccatt

SEQ ID NO: 9 - DNA, 27 bp, Mus musculus
ctgggagtaatggtgatcgagaagaaa

SEQ ID NO: 10 - protein, 9 aa, Mus musculus
LGAEAIFRL

SEQ ID NO: 11 - protein, 729 aa, Mus musculus
MLFNSVLRQPQLGVLRNGWSSHYPLQSLLSGYQCNCNDEHTSYGETGVPVPPFGCTFCTAPSMEHILAVANEEGFVRLYNTESQTSKKTCFKEWMAHWNAVFDLAWVPGELKLVTAAGDQTAKFWDVRAGELMGTCKGHQCSLKSVAFPKFQKAVFSTGGRDGNIMIWDTRCNKKDGFYRQVNQISGAHNTADKQTPSKPKKKQNSKGLAPAVDSQQSVTVVLFQDENTLVSAGAVDGIIKVWDLRKNYTAYRQEPIASKSFLYPGTSTRKLGYSSLVLDSTGSTLFANCTDDNIYMFNTTGLKTSPVAVFNGHQNSTFYVKSSLSPDDQFLISGSSDEAAYIWKVSMPWHPPTVLLGHSQEVTSVCWYPSDFTKIATCSDDNTLKIWRLNRGLEEKPGDKHSIVGWTSQKKKEVKACPVTVPSSQSTPAKAPRAKSSPSISSPSSAACTPSCAGDLPLPSSTPTFSVKTTPATTRSSVSRRGSISSVSPKPLSSFKMSLRNWVTRTPSSSPPVTPPASETKISSPRKALIPVSQKSSQADACSESRNRVKRRLDSSCLESVKQKCVKSCNCVTELDGQAESLRLDLCCLSGTQEVLSQDSEGPTKSSKTEGAGTSISEPPSPVSPYASEGCGPLPLPLRPCGEGSEMVGKENSSPENKNWLLAIAAKRKAENSSPRSPSSQTPSSRRQSGKTSPGPVTITPSSMRKICTYFRRKTQDDFCSPEHSTEL

SEQ ID NO: 12 - protein, 730 aa, Homo sapiens
MLFNSVLRQPQLGVLRNGWSSQYPLQSLLTGYQCSGNDEHTSYGETGVPVPPFGCTFSSAPNMEHVLAVANEEGFVRLYNTESQSFRKKCFKEWMAHWNAVFDLAWVPGELKLVTAAGDQTAKFWDVKAGELIGTCKGHQCSLKSVAFSKFEKAVFCTGGRDGNIMVWDTRCNKKDGFYRQVNQISGAHNTSDKQTPSKPKKKQNSKGLAPSVDFQQSVTVVLFQDENTLVSAGAVDGIIKVWDLRKNYTAYRQEPIASKSFLYPGSSTRKLGYSSLILDSTGSTLFANCTDDNIYMFNMTGLKTSPVAIFNGHQNSTFYVKSSLSPDDQFLVSGSSDEAAYIWKVSTPWQPPTVLLGHSQEVTSVCWCPSDFTKIATCSDDNTLKIWRLNRGLEEKPGGDKLSTVGWASQKKKESRPGLVTVTSSQSTPAKAPRVKCNPSNSSPSSAACAPSCAGDLPLPSNTPTFSIKTSPAKARSPINRRGSVSSVSPKPPSSFKMSIRNWVTRTPSSSPPITPPASETKIMSPRKALIPVSQKSSQAEACSESRNRVKRRLDSSCLESVKQKCVKSCNCVTELDGQVENLHLDLCCLAGNQEDLSKDSLGPTKSSKIEGAGTSISEPPSPISPYASESCGTLPLPLRPCGEGSEMVGKENSSPENKNWLLAMAAKRKAENPSPRSPSSQTPNSRRQSGKTLPSPVTITPSSMRKICTYFHRKSQEDFCGPEHSTEL

SEQ ID NO: 13 - DNA, 937 bp, Mus musculus
ttaaaagggagatggaaaggactccaatccaattaggggcagaagctatctttagactagtactaatgtggtgagtatcctgcctcacttaacatccttaacctagcctgtgcttcagggaaaggaaacaaaagatagatatataaaggctatttactctgcaagccaaaccaaaattttggtacagaaaatgacaaagtttctttttctcaatagtaggaatgttctgtgttcctagccaaaaatttgtcatcagtgtccctgtggctcccatctgtctttcctgacccccatgattacttttcactgacaatataaatcttaaaaagctttgagaccagtgtgctgatctagttttttaagttttgggttttgttgttgtgttttggttttggtgtttttacgttttattgacttacaagctttcactatgtatcccagactggctttgaactcaggaccttcctgccttagtctcccaggtgctgagattgtggggctgtgtcactatattacaactaatatttatcagaaaaatgttccttttttggaagaaaatgaagaaagtgctgtttttgcacttatcaattgaggggctgattttgattcgctcaagaaaatagcagttttaacctagacatttgctgtggggacttggaactttaacttggggtgtcgctaactctacgctttcttctaggtcaccattactcccagctccatgaggaagatatgtacatactttcgtagaaagactcaagatgacttctgcagtcctgaacactcaactgaattatagatgctaatctgaagttcattgaacttgggtctacaaagattttttaaaaaggctttaaaattctggtctttaagaaaatggtcatcttttcattttggaaaaagtccttctactctttagcagacctagtcaggcccc

SEQ ID NO: 14 - protein, 20 aa, Mus musculus
MERTPIQLGAEAIFRLVLMW

SEQ ID NO: 15 - protein, 19 aa, Mus musculus
MERTPIQLGAEAIFRLVLM

SEQ ID NO: 16 - protein, 9 aa, Mus musculus
MERTPIQLG

SEQ ID NO: 17 - protein, 9 aa, Mus musculus
ERTPIQLGA

SEQ ID NO: 18 - protein, 9 aa, Mus musculus
RTPIQLGAE

SEQ ID NO: 19 - protein, 9 aa, Mus musculus
TPIQLGAEA

SEQ ID NO: 20 - protein, 9 aa, Mus musculus
PIQLGAEAI

SEQ ID NO: 21 - protein, 9 aa, Mus musculus
IQLGAEAIF

SEQ ID NO: 22 - protein, 9 aa, Mus musculus
QLGAEAIFR

SEQ ID NO: 23 - protein, 9 aa, Mus musculus
GAEAIFRLV

SEQ ID NO: 24 - protein, 9 aa, Mus musculus
AEAIFRLVL

SEQ ID NO: 25 - protein, 9 aa, Mus musculus
EAIFRLVLM

SEQ ID NO: 26 - protein, 9 aa, Mus musculus
  MISC_FEATURE (1)..(1): Xaa = any amino acid
XGPXXXXXL

SEQ ID NO: 27 - protein, 9 aa, Mus musculus
  MISC_FEATURE (1)..(1): Xaa = any amino acid
XGAXXXXXL

SEQ ID NO: 28 - DNA, 4221 bp, Homo sapiens
cgataacgatttgtgttgtgagaggcgcaagctgcgatttctgctgaacttggaggcatttctacgacttttctctcagctgaggcttttcctccgaccctgatgctcttcaattcggtgctccgccagccccagcttggcgtcctgagaaatggatggtcttcacaataccctcttcaatcccttctgactggttatcagtgcagtggtaatgatgaacacacttcttatggagaaacaggagtcccagttcctccttttggatgtaccttctcttctgctcccaatatggaacatgtactagcagttgccaatgaagaaggctttgttcgattgtataacacagaatcacaaagtttcagaaagaagtgcttcaaagaatggatggctcactggaatgccgtctttgacctggcctgggttcctggtgaacttaaacttgttacagcagcaggtgatcaaacagccaaattttgggacgtaaaagctggtgagctgattggaacatgcaaaggtcatcaatgcagcctcaagtcagttgccttttctaagtttgagaaagctgtattctgtacgggtggaagagatggcaacattatggtctgggataccaggtgcaacaaaaaagatgggttttataggcaagtgaatcaaatcagtggagctcacaatacctcagacaagcaaaccccttcaaaacccaagaagaaacagaattcaaaaggacttgctccttctgtggatttccagcaaagtgttactgtggtcctctttcaagacgagaataccttagtctcagcaggagctgtggatgggataatcaaagtatgggatttacgtaagaattatactgcttatcgacaagaacccatagcatccaagtctttcctgtacccaggtagcagcactcgaaaacttggatattcaagtctgattttggattccactggctctactttatttgctaattgcacagacgataacatctacatgtttaatatgactgggttgaagacttctccagtggctattttcaatggacaccagaactctaccttttatgtaaaatccagccttagtccagatgaccagtttttagtcagtggctcaagtgatgaagctgcctacatatggaaggtctccacaccctggcaacctcctactgtgctcctgggtcattctcaagaggtcacgtctgtgtgctggtgtccatctgacttcacaaagattgctacctgttctgatgacaatacactaaaaatctggcgcttgaatagaggcttagaggagaaaccaggaggtgataaactttccacggtgggttgggcctctcagaagaaaaaagagtcaagacctggcctagtaacagtaacgagtagccagagtactcctgccaaagcccccagggtaaagtgcaatccatccaattcttccccgtcatccgcagcttgtgccccaagctgtgctggagacctccctcttccttcaaatactcctacgttctctattaaaacctctcctgccaaggcccggtctcccatcaacagaagaggctctgtctcctccgtctctcccaagccaccttcatctttcaagatgtcgattagaaactgggtgacccgaacaccttcctcatcaccacccatcactccacctgcttcggagaccaagatcatgtctccgagaaaagcccttattcctgtgagccagaagtcatcccaagcagaggcttgctctgagtctagaaatagagtaaagaggaggctagactcaagctgtctggagagtgtgaaacaaaagtgtgtgaagagttgtaactgtgtgactgagcttgatggccaagttgaaaatcttcatttggatctgtgctgccttgctggtaaccaggaagaccttagtaaggactctctaggtcctaccaaatcaagcaaaattgaaggagctggtaccagtatctcagagcctccgtctcctatcagtccgtatgcttcagaaagctgtggaacgctacctcttcctttgagaccttgtggagaagggtctgaaatggtaggcaaagagaatagttccccagagaataaaaactggttgttggccatggcagccaaacggaaggctgagaatccatctccacgaagtccgtcatcccagacacccaattccaggagacagagcggaaagacattgccaagcccggtcaccatcacgcccagctccatgaggaaaatctgcacatacttccatagaaagtcccaggaggacttctgtggtcctgaacactcaacagaattatagattctaatctgagtgagttactgagctttggtccactaaaacaagctgagctttggtccactaaaacaagatgaaaaatacaagagtgactctataactctggtctttaagaaagctgccttttcatttttagacaaaatcttttcaacgctgaaatgtacctaatctggttctactaccataatgtatatgcagcttcccgaggatgaatgctgtgtttaaatttcataaagtaaatttgtcactctagcattttgaatgaatagtcttcactttttaaattattcatcttctctataataatgacatcccagttcatggaggcaaaaaacaagtttcttgttatcctgaaactttctatgctcagtggaaagtatctgccagccacagcatgaggcctgtgaaggctgactgagaaatcctctgctgaagacccctggttctgttctgcctccaacatgtataattttatttgaaatacataatcttttcactatgcttttgtggggttttttttaagtatgtgtaaaaatgtgatgctcagataagtacatttatatcagttcagtgttaaaatgcagtctcttgagttaaagtcatctttattttaaatgcagtgataaatgtcaactcttcggagaaactaggagaacaacaacagaaagctgtgtttgtcttttttctctcaaatatatctcccgtatgagatttcaggtccccatgttttcaccaagcaatctgctatgtcagccaacccaacatcactttctacaggaggttatgatttttgccatttactagaggaagatgttttatgaaatcaatttggggtttgaattcaggtgcagtcatcagttctttaggggctgcaatgttttaaaaaaaataagtcatcagattttaagaaaaaagtgatgatttcttattgatatttttgtaacagaatatagctcttaactgaaaatccagaaccagaaacataaatcttgagtttcttttcatgtacataaaaagcaatagccttttagtatagatagccctgagccaaaaagtaatagaattttctctagatatttaatacagagagtgtatagactgactctaagttaataatgtgcaaaatatcttaaacatccctccccttattcaacaattatgtatcagtgatcttgaaccattgttttatatttttcacctttgtaacctcatggaaagaggctttacatactttctatgtactatttacttagaagggagcccccttccagtcatgaaacttcatttgttttatccatatccctgaggactgtgtagactttatgtcagttctgtgtagactttatgtcagtttttgtcattatttgaaaatctattctgacaactttttaattcctttgatcttataagttaaagctgtaacaactgaaattgcatggatcaagtaagcatagttttatccagggagaaaaataaaaggaagccatagaattgctctggtcaaaaccaagcacaccatagccttaactgaatatttaggaaatctgcctaatctgcttatatttggtgtttgttttttgactgttgggctttgggaagatgttatttatgaccaatatctgccagtaacgctgtttatctcacttgctttgaaagccaatgggggaaaaaaatccatgaaaaaaaaaagattgataaagtagatgattttgtttgtatccctacccatctcctggcagccctactgagtgaaattgggatacatttggctgtcagaaattataccgagtctactgggtataacatgtctcacttggaaagctagtacttttaaatgggtgccaaaggtcaactgtaatgagataattatccctgcctgtgtccatgtcagactttgagctgatcctgaataataaagccttttacctt